(19) United States
(12) Patent Application Publication
(10) Pub. No.: US 2008/0221129 A1
(43) Pub. Date: Sep. 11, 2008

(54) New compounds

(76) Inventors: Thomas Lundback, Trangsund (SE); Isabel Climent-Johansson, Stockholm (SE); Jan Vagberg, Sollentuna (SE); Auri Linden, Stockholm (SE); Jonas Nilsson, Ingaro (SE); Marie Wiik, Uppsala (SE); Ulf Bremberg, Uppsala (SE)

Correspondence Address: EDWARDS ANGELL PALMER & DODGE LLP, P.O. BOX 55874, BOSTON, MA, 02205, US

(21) Appl. No.: 11/825,431

(22) Filed: Jul. 6, 2007

Related U.S. Application Data
(60) Provisional application No. 60/860,677, filed on Nov. 21, 2006.

(30) Foreign Application Priority Data
Jul. 7, 2006 (SE) 0601511-9

(51) Int. Cl. A61K 31/519; C07D 487/04; A61P 9/00; A61P 3/04; A61P 25/00; A61P 17/00

(52) U.S. Cl. 514/259.3; 544/281

(57) ABSTRACT

The present invention relates to compounds of the formula (I):

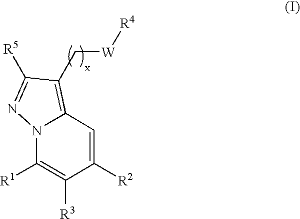

including pharmaceutically acceptable salts, solvates, hydrates, geometrical isomers, tautomers, optical isomers, and N-oxides thereof, said compounds being useful as inhibitors of stearoyl-CoA desaturase (SCD). The invention further relates to the use of compounds of the formula (I) for treatment of medical conditions in which the modulation of SCD activity is beneficial, such as cardiovascular diseases, obesity, non-insulin-dependent diabetes mellitus, hypertension, neurological diseases, immune disorders, cancer, essential fatty acid deficiency, acne, psoriasis, rosacea or other skin conditions.

### CROSS REFERENCE TO RELATED APPLICATIONS

This application claims benefit of U.S. Provisional Application No. 60/860,677, filed Nov. 21, 2006; and Swedish Application No. 0601511-9, filed Jul. 7, 2006. the entire content of each of these applications are herein incorporated by reference.

### TECHNICAL FIELD

The present invention relates to compounds of the formula (I), said compounds being useful as inhibitors of stearoyl-CoA desaturase (SCD) activity. The invention further relates to the use of compounds of the formula (I) for treatment of medical conditions in which the modulation of SCD activity is beneficial, such as cardiovascular diseases, obesity, non-insulin-dependent diabetes mellitus, hypertension, neurological diseases, immune disorders, cancer and various skin diseases.

### BACKGROUND ART

The lipid composition of cellular membranes is regulated to maintain membrane fluidity. A key enzyme involved in this process is the microsomal stearoyl-CoA desaturase (SCD; Δ9-desaturase; EC 1.14.99.5), which is the rate-limiting enzyme in the cellular synthesis of monounsaturated fatty acids from saturated fatty acids [see e.g. Ntambi (1999) J. Lipid Res. 40, 1549 for a review]. The principal products of SCD are oleoyl-CoA and palmitoleoyl-CoA, which are formed by desaturation of stearoyl-CoA and palmitoyl-CoA, respectively. A proper ratio of saturated to monounsaturated fatty acids contributes to membrane fluidity. Alterations in this ratio have been implicated in various disease states including cardiovascular disease, obesity, non-insulin-dependent diabetes mellitus, hypertension, neurological diseases, immune disorders, cancer and various skin diseases (Ntambi (1999) J. Lipid Res. 40, 1549). The regulation of SCD, the expression and activity of which is known to be sensitive to e.g. dietary changes and hormonal balance, is therefore of considerable physiological importance.

Several mammalian SCD genes have been cloned. Four SCD isoforms, SCD1 through SCD4, have been identified in mouse. In contrast, only two isoforms are known in rat and man. The sequence of human SCD1 from liver was first deposited in June 1997 (GenBank accession number Y13647) and the full-length cloning of human SCD1 is (GenBank accession number Y13647) and the full-length cloning of human SCD1 is later described in WO 00/09754 and in Zhang et al. (1999) Biochem. J. 340, 255. The other human SCD isoform has been named SCD5 because it bears little sequence homology to alternate mouse or rat isoforms (WO 02/26944; Zhang et al. (2005) Biochem J. 388, 135; Wang et al. (2005) Biochem. Biophys. Res. Comm. 332, 735).

Early studies in rodents demonstrated that insulin as well as carbohydrate rich diets are key components in the upregulation of hepatic SCD activity [Oshino and Sato (1972) Arch. Biochem. Biophys. 149, 369; Prasad and Joshi (1979) J. Biol. Chem. 254, 997; Waters and Ntambi (1994) J. Biol. Chem. 269, 27773]. Fructose appears to play a key role in this process since this carbohydrate, contrary to glucose, not only upregulates hepatic SCD activity but also corrects the defective lipogenesis that appears in diabetic animals (see above cited references and references therein). Later studies showed that the expression of SCD1, the major SCD isoform in hepatocytes, is a crucial component in the fructose-mediated elevation of lipogenic enzymes [Miyazaki et al. (2004) J. Biol. Chem. 279, 25164], demonstrating a key role of this enzyme in hepatic lipogenesis.

There were also observations of elevated SCD activity in animal models of type 2 diabetes and obesity [see e.g. Enser (1975) Biochem. J. 148, 551; Legrand and Hermier (1992) Int. J. Obes. Relat. Metab Disord. 16, 289; Jones et al. (1996) Am. J. Physiol. 271, E44] and increased SCD activity was also shown to be associated with obesity in man [Pan et al. (1994) J. Nutr. 124, 1555], which led to descriptions of the potential role of SCD activity in type 2 diabetes and obesity amongst other diseases [Ntambi J M. (1999) J. Lipid Res. 40, 1549]. SCD1 appeared to be of primary interest based on the selective suppression of this isoform in differentiating preadipocytes by thiazolidinediones, data that were strengthened by the suppression of SCD1 in tissues of metabolic interest in vivo [Kim et al. (2000) In: Adipocyte Biology and Hormone Signaling, 27th Steenbock Symposium, Madison, Wis., June, 1999 (J. M. Ntambi, ed.), IOS Press, The Netherlands, pp. 69].

More recent studies based on animal models in which SCD1 levels are suppressed either by means of genetic ablation or by anti-sense treatment have confirmed a key role of SCD1 in the regulation of lipid synthesis versus oxidation as well as for the development of diet-induced obesity [Miyazaki et al. (2000) J. Biol. Chem. 275, 30132; WO 01/62954; Ntambi et al. (2002) Proc. Natl. Acad. Sci. USA 99, 11482; Cohen et al. (2002) Science 297, 240; Jiang et al. (2005) J. Clin. Invest. 115, 1030; Gutierrez-Juarez et al. (2006) J. Clin. Invest. 116, 1686]. The interest in SCD activity as a potential target for the development of anti-obesity treatments has thus increased significantly, prompted also by additional reports on the correlation of SCD1 activity with circulating triglyceride levels in mice as well as man [WO 01/62954; Attie et al. (2002) J. Lipid Res. 43, 1899] as well as confirming observations of elevated SCD activity in the muscles of obese people [Hulver et al. (2005) Cell Metab. 2, 251].

Besides the above described findings, both asebia mice carrying a deletion in the SCD1 gene (Zheng et al. (1999) Nature Genet. 23, 268) and SCD1 knock-out mice (Miyazaki et al. (2001) J. Nutr. 131, 2260) develop skin and eye abnormalities. These changes include hair loss as well as atrophy of the sebaceous and meibomian glands. It is therefore believed that modulation of SCD activity can be of importance in the treatment of disease states that are associated with changes in the lipid composition in these tissues and their lipid secretions as well as changes in the composition of circulating lipids that impact these tissues (see e.g. Ntambi (1999) J. Lipid Res. 40, 1549 for a general description and United States Patent 20020151018 for a more specific description). Skin diseases where it could be of relevance to apply a modulator of SCD activity include but are not restricted to e.g. essential fatty acid deficiency, acne, psoriasis and rosacea. Based on the above described phenotypes other potential applications of a SCD modulator involve a selective suppression or stimulation of hair growth (see e.g. European patent application EP1352627 A2).

It is furthermore clear for anyone skilled in the art that the desired distribution of these modulators may depend on the therapeutic indication or disease state or other application of the compounds described herein. Hence for the treatment of metabolic diseases such as type 2 diabetes and obesity, it may be desirable not to impact skin glands, hair or eyes in a negative way, i.e. such as what is observed in the above described mouse models that lack SCD1 expression. Pharmacological modulation of SCD1 activity by means of anti-sense mediated inhibition shows beneficial effects on type 2 diabetes and obesity parameters, without a negative impact on hair or skin [Jiang et al. (2005) J. Clin. Invest. 115, 1030; Gutierrez-Juarez et al. (2006) J. Clin. Invest. 116, 1686]. It is possible that this results from a reduced level of inhibition of SCD1 expression compared to the homozygous SCD1 knock-outs, but it may also be caused by the limited tissue distribution that is typically seen with anti-sense based inhibitors. On the contrary, for treatments of skin or hair diseases it may be desirable to ensure exposure in these tissues while limiting systemic exposure, such that e.g. direct application to the skin may be preferable. It is thus clear that depending on the respective tissue distribution profiles, whether caused by their intrinsic properties or by the use of various forms of administrations or formulations, SCD activity modulators will be suitable for different therapeutic indications.

The above described data serve to illustrate the validity of modulating stearoyl-CoA desaturase activity for treatment of disorders and diseases that include but are not restricted to those related to the metabolic syndrome, e.g. type 2 diabetes, obesity, non-alcoholic fatty liver disease and more. It is also described in the above cited literature that more than one isoform of SCD exists, the numbers and identities of which differ between species. The majority of findings as outlined above and in the cited references refers to SCD1, but the contributions made by SCD5 to the metabolism in man are less well understood. Depending on what disorder or disease a treatment is aimed at the modulation of the stearoyl-CoA desaturase activity may therefore involve the modulation of both or either of these activities. Consequently, there is a need for identifying molecules that modulate SCD activity and are potentially useful for the treatment of e.g. type 2 diabetes, coronary artery disease, atherosclerosis, heart disease, cerebrovascular disease, essential fatty acid deficiency, acne, psoriasis, rosacea, or for the treatment of excessive hair growth.

Substituted pyrazolopyrimidine compounds are known in the art, see e.g. U.S. patent application Ser. No. 11/244,628 (Publication No. 2006/0094706). However, it has not previously been shown that such compounds are capable of modulating SCD activity.

### DISCLOSURE OF THE INVENTION

It has surprisingly been shown that compounds of the formulae herein (e.g., (I-III)) are active as inhibitors of SCD activity. As such they are potentially useful for modulating SCD activity and thereby can serve to regulate lipid levels and composition in mammals. As such they are potentially useful in the treatment of SCD related diseases such as cardiovascular diseases, obesity, non-insulin-dependent diabetes mellitus, hypertension, neurological diseases, immune disorders, cancer and various skin diseases.

Consequently, the invention relates to a compound of formula (I), for use in therapy,

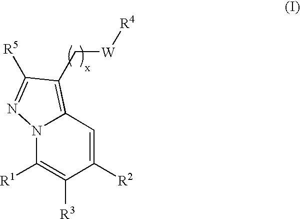

including pharmaceutically acceptable salts, solvates, hydrates, geometrical isomers, tautomers, optical isomers, and N-oxides thereof, wherein:
x is 0 or 1;
W is a direct bond, —C(O)N(R⁶)—, —N(R⁶)C(O)—, —C(O)O—, —OC(O)—, —O—, —N(R⁶)C(O)N(R⁶)—, —N(R⁶)—; wherein each R⁶ is independently hydrogen, C₁-C₃ alkyl, or C₃-C₈ alkoxyalkyl;
One of R¹, R² and R³ is Y-R¹⁸, and the other two are independently selected from the group consisting of hydrogen, C₁-C₃ alkyl and C₁-C₃ fluoroalkyl;
Y is selected from the group consisting of —S—, —O—, and C₁-C₃ alkylene, wherein C₁-C₃ alkylene is optionally monosubstituted with hydroxy or oxo, or is partly or fully fluorinated;
R¹⁸ is aryl or heteroaryl, which is optionally substituted in one or more positions;
R⁴ is selected from the group consisting of hydrogen, C₁-C₆ alkyl, C₂-C₆ alkenyl, C₃-C₆-alkynyl, C₁-C₆ fluoroalkyl, C₃-C₈ alkylthioalkyl, C₃-C₆ cyanoalkyl, C₈-C₁₂ arylalkyl, C₃-C₆ cycloalkyl, C₃-C₈ heteroaryl, aryl, C₅-C₁₀ heteroarylalkyl, C₄-C₆-heterocyclylalkyl and C₃-C₉ heterocyclyl, provided that said heterocyclyl is bonded via a ring carbon;
or R⁴ is C₁₋₆ alkylene-V—R⁷;
wherein V is selected from the group consisting of —N(R¹⁵)—, —C(O)N(R¹⁵)—, —C(O)O—, —OC(O)—, —C(O)—, —O—, —N(R¹⁵)C(O)—, —N(R¹⁵)C(O)N(R¹⁵)—, —S—, —S(O)—, —S(O)₂—, —S(O)₂N(R¹⁵)— and —N(R¹⁵)S(O)₂—;
and wherein each R⁷ and each R¹⁵ are independently selected from the group consisting of hydrogen, C₁-C₅ alkyl, hydroxy-C₁-C₅ alkyl, aryl-C₁-C₅ alkyl, heteroaryl-C₁-C₅ alkyl, heteroaryl, heterocyclyl, C₄-C₈ cycloalkylalkyl, C₃-C₈ cycloalkyl and C₁-C₅ fluoroalkyl, provided that when V is selected from —S—, —S(O)— or —S(O)₂—, R⁷ is not hydrogen;
   or R⁴ and R⁶ together form a C₃-C₅ heterocyclyl ring; and
   R⁵ is hydrogen or C₁-C₃ alkyl;
   provided that the said compound is not selected from the group consisting of:
N-cyclopentyl-5,7-dimethyl-6-(2,4,6-trimethylbenzyl)-pyrazolo[1,5-a]pyrimidine-3-carboxamide; and
6-(4-chlorobenzyl-5,7-dimethyl-N-(1-methylethyl)-pyrazolo[1,5-a]pyrimidine-3-carboxamide.
   Preferred compounds of the formula I include those wherein:
   x is 0 and W is —C(O)NH—,
   R¹ is methyl;
   R² is methyl;
   R³ is C₇-C₁₂ arylalkyl;
   R⁴ is C₃-C₈ alkoxyalkyl, C₂-C₆ hydroxyalkyl, C₃-C₈ alkylthioalkyl, or C₄-C₆ heterocyclylalkyl;
   R⁴ is C₁-C₆ alkylene-V—R⁷;
   wherein V is selected from the group consisting of —N(R¹⁵)C(O)—, —C(O)N(R¹⁵)—, —O— and —S(O)—,
   and wherein each R⁷ and each R¹⁵ are independently selected from the group consisting of hydrogen, C₁-C₅ alkyl, hydroxy-C₁-C₅ alkyl, C₂-C₅ fluoroalkyl, C₃-C₆ cycloalkyl and heteroaryl;
   R⁴ and an R⁶ together form a C₃-C₅ heterocyclyl ring; and/or
   R⁵ is H.

More preferred compounds of formula I include those, wherein x is 0 or 1;

W is a direct bond, —C(O)N(R⁶)—, —N(R⁶)C(O)—, —C(O)O—, —OC(O)—, —N(R⁶)C(O)N(R⁶)—, N(R⁶)—;
wherein each R⁶ is independently hydrogen, C₁-C₃ alkyl, or C₃-C₈ alkoxyalkyl;
R¹ and R² are each independently selected from the group consisting of hydrogen, C₁-C₃ alkyl, and C₁-C₃ fluoroalkyl;
R³ is C₇-C₁₂ arylalkyl or C₃-C₁₀ heteroarylalkyl;
R⁴ is selected from the group consisting of hydrogen, C₁-C₆ alkyl, C₂-C₆ alkenyl, C₃-C₆ alkynyl, C₁-C₆ fluoroalkyl, C₃-C₈ alkoxyalkyl, C₁-C₆ hydroxyalkyl, C₃-C₈ alkylthioalkyl, C₃-C₆ cyanoalkyl, C₈-C₁₂ arylalkyl, C₃-C₆ cycloalkyl, C₃-C₈ heteroaryl, aryl, C₄-C₆ heterocyclylalkyl, and C₃-C₉ heterocyclyl, provided that said heterocyclyl is bonded via a ring carbon;
or R⁴ is C₁₋₆ alkylene-V—R⁷;
wherein V is selected from the group consisting of —N(R¹⁵)—, —C(O)N(R¹⁵)—, —C(O)O—, and —OC(O)—, —C(O)—, —N(R¹⁵)C(O)—, —N(R¹⁵)C(O)N(R¹⁵)—, —S(O)—, —S(O)₂—, —S(O)₂N(R¹⁵)—, —N(R¹⁵)S(O)₂—;
and wherein each R⁷ and each R¹⁵ are independently selected from the group consisting of hydrogen, C₁-C₅ alkyl, C₄-C₈ cycloalkylalkyl, C₃-C₈ cycloalkyl and C₁-C₅ fluoroalkyl, provided that when V is selected from —S(O)— or —S(O)₂—, R⁷ is not hydrogen;
   or R⁴ and an R⁶ together form a C₃-C₅ heterocyclyl ring; and
   R⁵ is hydrogen or C₁-C₃ alkyl.

Particularly preferred compounds for use in therapy according to the invention are compounds having the Formula II, including pharmaceutically acceptable salts, solvates, hydrates, geometrical isomers, tautomers, optical isomers, and N-oxides thereof,

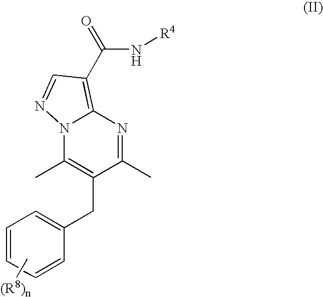

wherein:
R⁴ is as defined for formula I,
n is 0, 1, 2 or 3; and
each R⁸ is independently selected from the group consisting of fluoro, chloro, bromo, methyl, ethyl, methoxy, ethoxy, hydroxy, hydroxymethyl, trifluoromethyl, trifluoromethoxy, methylthio, trifluoromethylthio and benzyloxy, or two substituents R⁸ together form a saturated or unsaturated, aliphatic or heterocyclic ring;
provided that the said compound is not selected from the group consisting of:
N-cyclopentyl-5,7-dimethyl-6-(2,4,6-trimethylbenzyl)-pyrazolo[1,5-a]pyrimidine-3-carboxamide; and
6-(4-chlorobenzyl-5,7-dimethyl-N-(1-methylethyl)-pyrazolo[1,5-a]pyrimidine-3-carboxamide.

Preferred compounds of formula II include those wherein R⁴ is C₃-C₈ alkoxyalkyl, C₂-C₆ hydroxyalkyl, C₃-C₈ alkylthioalkyl, or C₄-C₆ heterocyclylalkyl.

More preferred compounds of formula II include those wherein R⁴ is 2-methoxyethyl, 3-ethoxypropyl, 3-isopropoxypropyl, tetrahydrofuran-2-ylmethyl, or 2-(1,3-dioxolan-2-yl)ethyl.

Other preferred compounds of formula II include those wherein R⁴ is

C₁-C₆ alkylene-V—R⁷;
wherein V is selected from the group consisting of —N(R¹⁵)C(O)—, —C(O)N(R¹⁵)—, —O— and —S(O)—,
and wherein each R⁷ and each R¹⁵ are independently selected from the group consisting of hydrogen, C₁-C₅ alkyl, hydroxy-C₁-C₅ alkyl, C₂-C₅ fluoroalkyl, and C₃-C₆ cycloalkyl and heteroaryl.

Other preferred compounds of formula II include those wherein

R⁴ is —(CH₂)ₚ—NHC(O)R⁹
wherein R⁹ is C₁-C₃ alkyl and p is 2, 3, or 4;
   or R⁴ is —(CH₂)_{z}—C(O)NR¹⁷R¹⁷;
wherein each R¹⁷ is independently hydrogen or C₁-C₃ alkyl; and z is 1 or 2.

More preferred compounds of formula II include those, wherein R⁴ is C₃-C₈ alkoxyalkyl, C₂-C₆ hydroxyalkyl, C₃-C₈ alkylthioalkyl, or C₄-C₆ heterocyclylalkyl;

R⁴ is C₁-C₆ alkylene-V—R⁷;
wherein V is selected from the group consisting of —N(R¹⁵)C(O)— and —C(O)N(R¹⁵),
and wherein each R⁷ and each R¹⁵ are independently selected from the group consisting of hydrogen, C₁-C₅ alkyl, C₂-C₅ fluoroalkyl, and C₃-C₆ cycloalkyl;
   or R⁴ and R⁶ together form a C₃-C₅ heterocyclyl ring; and
   each R⁸ is independently selected from the group consisting of fluoro, chloro, bromo, methyl, ethyl, methoxy, ethoxy, hydroxy, trifluoromethyl, hydroxymethyl and methylthio.

Specific preferred compounds for use in therapy according to the invention are those selected from the group consisting of:
6-Benzyl-N-(2-methoxyethyl)-5,7-dimethylpyrazolo[1,5-a]pyrimidine-3-carboxamide;
6-(3-Bromobenzyl)-N-(2-methoxyethyl)-5,7-dimethylpyrazolo[1,5-a]-pyrimidine-3-carboxamide;
6-(2-Fluorobenzyl)-N-(2-methoxyethyl)-5,7-dimethylpyrazolo[1,5-a]-pyrimidine-3-carboxamide;
6-(2-Fluorobenzyl)-N-(3-methoxypropyl)-5,7-dimethylpyrazolo[1,5-a]-pyrimidine-3-carboxamide;
6-(2-Bromobenzyl)-N-(3-methoxypropyl)-5,7-dimethylpyrazolo[1,5-a]-pyrimidine-3-carboxamide;
6-(3-Bromobenzyl)-5,7-dimethyl-N-(tetrahydrofuran-2-ylmethyl)pyrazolo-[1,5-a]pyrimidine-3-carboxamide;
6-(Mesitylmethyl)-N-(2-methoxyethyl)-5,7-dimethylpyrazolo[1,5-a]-pyrimidine-3-carboxamide;
N-(2-Methoxyethyl)-5,7-dimethyl-6-(2-methylbenzyl)pyrazolo[1,5-a]-pyrimidine-3-carboxamide;
6-(2,5-Dimethylbenzyl)-N-(2-methoxyethyl)-5,7-dimethylpyrazolo[1,5-a]-pyrimidine-3-carboxamide;
6-(4-Chlorobenzyl)-N-(2-methoxyethyl)-5,7-dimethylpyrazolo[1,5-a]-pyrimidine-3-carboxamide;
6-(2-Chlorobenzyl)-N-(2-methoxyethyl)-5,7-dimethylpyrazolo[1,5-a]-pyrimidine-3-carboxamide;
6-(3-Chlorobenzyl)-N-(2-methoxyethyl)-5,7-dimethylpyrazolo[1,5-a]-pyrimidine-3-carboxamide;
5,7-Dimethyl-6-(2-methylbenzyl)-N-(tetrahydrofuran-2-ylmethyl)pyrazolo-[1,5-a]pyrimidine-3-carboxamide;
6-(2,5-Dimethylbenzyl)-5,7-dimethyl-N-(tetrahydrofuran-2-ylmethyl)-pyrazolo[1,5-a]pyrimidine-3-carboxamide;
Methyl 6-(3-bromobenzyl)-5,7-dimethylpyrazolo[1,5-a]pyrimidine-3-carboxylate;
6-(4-Fluorobenzyl)-5,7-dimethyl-N-(tetrahydrofuran-2-ylmethyl)pyrazolo[1,5-a]pyrimidine-3-carboxamide;
6-(4-Chlorobenzyl)-N-(3-methoxypropyl)-5,7-dimethylpyrazolo[1,5-a]-pyrimidine-3-carboxamide;
6-(4-Chlorobenzyl)-N-(3-ethoxypropyl)-5,7-dimethylpyrazolo[1,5-a]-pyrimidine-3-carboxamide;
6-(2-Chlorobenzyl)-N-(3-methoxypropyl)-5,7-dimethylpyrazolo[1,5-a]-pyrimidine-3-carboxamide;
6-(3-Chlorobenzyl)-N-(3-methoxypropyl)-5,7-dimethylpyrazolo[1,5-a]-pyrimidine-3-carboxamide;
6-(3-Chlorobenzyl)-N-(3-ethoxypropyl)-5,7-dimethylpyrazolo[1,5-a]-pyrimidine-3-carboxamide;
6-(2-Chlorobenzyl)-5,7-dimethyl-N-(tetrahydrofuran-2-ylmethyl)pyrazolo-[1,5-a]pyrimidine-3-carboxamide;
6-(2-Chloro-4-fluorobenzyl)-5,7-dimethyl-N-(tetrahydrofuran-2-ylmethyl)-pyrazolo[1,5-a]pyrimidine-3-carboxamide;
6-(2-Chloro-6-fluorobenzyl)-5,7-dimethyl-N-(tetrahydrofuran-2-ylmethyl)-pyrazolo[1,5-a]pyrimidine-3-carboxamide;
N-(3-Ethoxypropyl)-5,7-dimethyl-6-(2-methylbenzyl)pyrazolo[1,5-a]-pyrimidine-3-carboxamide;
N-(3-Methoxypropyl)-5,7-dimethyl-6-(2-methylbenzyl)pyrazolo[1,5-a]-pyrimidine-3-carboxamide;
N-(3-Ethoxypropyl)-6-(3-methoxybenzyl)-5,7-dimethylpyrazolo[1,5-a]-pyrimidine-3-carboxamide;
6-(3-Methoxybenzyl)-N-(2-methoxyethyl)-5,7-dimethylpyrazolo[1,5-a]-pyrimidine-3-carboxamide;
6-(3-Methoxybenzyl)-N-(3-methoxypropyl)-5,7-dimethylpyrazolo[1,5-a]-pyrimidine-3-carboxamide;
6-(3-Cyanobenzyl)-N-(3-methoxypropyl)-5,7-dimethylpyrazolo[1,5-a]-pyrimidine-3-carboxamide;
N-(3-Ethoxypropyl)-6-(3-fluorobenzyl)-5,7-dimethylpyrazolo[1,5-a]-pyrimidine-3-carboxamide;
6-(3-Fluorobenzyl)-N-(2-methoxyethyl)-5,7-dimethylpyrazolo[1,5-a]-pyrimidine-3-carboxamide;
6-(3-Fluorobenzyl)-N-(3-methoxypropyl)-5,7-dimethylpyrazolo[1,5-a]-pyrimidine-3-carboxamide;
6-Benzyl-N-(3-ethoxypropyl)-5,7-dimethylpyrazolo[1,5-a]pyrimidine-3-carboxamide;
6-Benzyl-N-(3-methoxypropyl)-5,7-dimethylpyrazolo[1,5-a]pyrimidine-3-carboxamide;
N-[2-(Acetylamino)ethyl]-5,7-dimethyl-6-(2-methylbenzyl)pyrazolo[1,5-a]-pyrimidine-3-carboxamide;
N-(3-Isopropoxypropyl)-5,7-dimethyl-6-(2-methylbenzyl)pyrazolo[1,5-a]-pyrimidine-3-carboxamide;
N-(2-Amino-2-oxoethyl)-5,7-dimethyl-6-(2-methylbenzyl)pyrazolo[1,5-a]-pyrimidine-3-carboxamide;
6-(3-Chlorobenzyl)-5,7-dimethyl-N-(tetrahydrofuran-2-ylmethyl)pyrazolo-[1,5-a]pyrimidine-3-carboxamide;
6-(3-Chlorobenzyl)-5,7-dimethyl-N-[2-(methylthio)ethyl]pyrazolo[1,5-a]-pyrimidine-3-carboxamide;
N-[2-(Acetylamino)ethyl]-6-(3-methoxybenzyl)-5,7-dimethylpyrazolo[1,5-a]-pyrimidine-3-carboxamide;
N-(3-Isopropoxypropyl)-6-(3-methoxybenzyl)-5,7-dimethylpyrazolo[1,5-a]-pyrimidine-3-carboxamide;
6-(3-Methoxybenzyl)-5,7-dimethyl-N-(tetrahydrofuran-2-ylmethyl)pyrazolo-[1,5-a]pyrimidine-3-carboxamide;
N-[2-(Acetylamino)ethyl]-6-(3-fluorobenzyl)-5,7-dimethylpyrazolo[1,5-a]-pyrimidine-3-carboxamide;
6-(3-Fluorobenzyl)-N-(3-isopropoxypropyl)-5,7-dimethylpyrazolo[1,5-a]-pyrimidine-3-carboxamide;
N-[2-(1,3-Dioxolan-2-yl)ethyl]-6-(3-fluorobenzyl)-5,7-dimethylpyrazolo[1,5-a]pyrimidine-3-carboxamide;
N-[2-(Acetylamino)ethyl]-6-benzyl-5,7-dimethylpyrazolo[1,5-a]pyrimidine-3-carboxamide;
6-Benzyl-N-(3-isopropoxypropyl)-5,7-dimethylpyrazolo[1,5-a]pyrimidine-3-carboxamide;
6-(3-Bromobenzyl)-N-(3-isopropoxypropyl)-5,7-dimethylpyrazolo[1,5-a]-pyrimidine-3-carboxamide;
N-[2-(Acetylamino)ethyl]-6-(3-bromobenzyl)-5,7-dimethylpyrazolo[1,5-a]-pyrimidine-3-carboxamide;
N-(2-Amino-2-oxoethyl)-6-(3-bromobenzyl)-5,7-dimethylpyrazolo[1,5-a]pyrimidine-3-carboxamide;
6-(3-Bromobenzyl)-N-(2-hydroxyethyl)-5,7-dimethylpyrazolo[1,5-a]-pyrimidine-3-carboxamide;
6-(3-Bromobenzyl)-N-(2-tert-butoxyethyl)-5,7-dimethylpyrazolo[1,5-a]-pyrimidine-3-carboxamide;
6-(3-Bromobenzyl)-N-(2-isopropoxyethyl)-5,7-dimethylpyrazolo[1,5-a]-pyrimidine-3-carboxamide;
6-Benzyl-5,7-dimethyl-N-(tetrahydrofuran-2-ylmethyl)pyrazolo[1,5-a]-pyrimidine-3-carboxamide;
6-(3,4-dichlorobenzyl)-N-[2-(2-hydroxyethoxy)ethyl]-5,7-dimethylpyrazolo-[1,5-a]pyrimidine-3-carboxamide;
5,7-dimethyl-N-[3-(methylamino)-3-oxopropyl]-6-[3-(trifluoromethoxy)-benzyl]pyrazolo[1,5-a]pyrimidine-3-carboxamide;
N-{3-[(2-hydroxyethyl)amino]-3-oxopropyl}-5,7-dimethyl-6-[3-(trifluoro-methoxy)benzyl]pyrazolo[1,5-a]pyrimidine-3-carboxamide;
6-(3-chlorobenzyl)-N-(2-methoxyethyl)-2,5,7-trimethylpyrazolo[1,5-a]-pyrimidine-3-carboxamide;
6-(3-chloro-4-fluorobenzyl)-N-(3-methoxypropyl)-5,7-dimethylpyrazolo[1,5-a]pyrimidine-3-carboxamide;
N-(3-amino-3-oxopropyl)-6-(3-chloro-4-fluorobenzyl)-5,7-dimethylpyrazolo-[1,5-a]pyrimidine-3-carboxamide;
N-(3-methoxypropyl)-5,7-dimethyl-6-[3-(trifluoromethyl)benzyl]pyrazolo-[1,5-a]pyrimidine-3-carboxamide;
5,7-dimethyl-N-[2-(1-methyl-1H-imidazol-4-yl)ethyl]-6-[3-(trifluoromethyl)-benzyl]pyrazolo[1,5-a]pyrimidine-3-carboxamide;
N-(2-amino-2-oxoethyl)-5,7-dimethyl-6-[3-(trifluoromethyl)benzyl]pyrazolo-[1,5-a]pyrimidine-3-carboxamide;
N-(3-hydroxypropyl)-5,7-dimethyl-6-[3-(trifluoromethyl)benzyl]pyrazolo[1,5-a]pyrimidine-3-carboxamide;
6-(3,5-dichlorobenzyl)-N-(2-methoxyethyl)-5,7-dimethylpyrazolo[1,5-a]-pyrimidine-3-carboxamide;
N-(2-methoxyethyl)-5,7-dimethyl-6-[3-(trifluoromethoxy)benzyl]pyrazolo-[1,5-a]pyrimidine-3-carboxamide;
6-(2,5-dichlorobenzyl)-N-(2-methoxyethyl)-5,7-dimethylpyrazolo[1,5-a]-pyrimidine-3-carboxamide;
6-(4-bromobenzyl)-N-(2-methoxyethyl)-5,7-dimethylpyrazolo[1,5-a]-pyrimidine-3-carboxamide;
N-[2-(acetylamino)ethyl]-6-[3-(benzyloxy)benzyl]-5,7-dimethylpyrazolo[1,5-a]pyrimidine-3-carboxamide;
N-(2-methoxyethyl)-5,7-dimethyl-6-(2-naphthylmethyl)pyrazolo[1,5-a]-pyrimidine-3-carboxamide;
5,7-dimethyl-6-(3-methylbenzyl)-N-{2-[(pyrazin-2-ylcarbonyl)amino]ethyl}-pyrazolo[1,5-a]pyrimidine-3-carboxamide;
N-[2-(benzyloxy)ethyl]-5,7-dimethyl-6-[3-(trifluoromethoxy)benzyl]pyrazolo-[1,5-a]pyrimidine-3-carboxamide;
N-(3-amino-3-oxopropyl)-6-(3,5-dichlorobenzyl)-5,7-dimethylpyrazolo[1,5-a]pyrimidine-3-carboxamide;
5,7-dimethyl-N-{2-[(pyridin-3-ylcarbonyl)amino]ethyl}-6-[3-(trifluoro-methoxy)benzyl]pyrazolo[1,5-a]pyrimidine-3-carboxamide;
6-[4-fluoro-3-(trifluoromethoxy)benzyl]-N-[2-(2-hydroxyethoxy)ethyl]-5,7-dimethylpyrazolo[1,5-a]pyrimidine-3-carboxamide; and

6-[4-fluoro-3-(trifluoromethyl)benzyl]-N-[2-(2-hydroxyethoxy)ethyl]-5,7-dimethylpyrazolo[1,5-a]pyrimidine-3-carboxamide.

In another aspect, the invention provides novel compounds of the formula III:

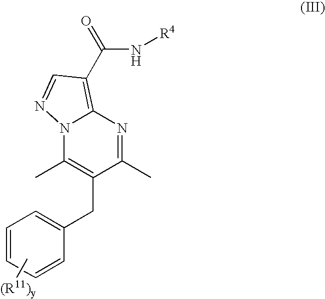

including pharmaceutically acceptable salts, solvates, hydrates, geometrical isomers, tautomers, optical isomers, and N-oxides thereof; wherein:
R¹⁰ is C₁₋₆alkylene-Z-R¹²;
wherein Z is selected from the group consisting of —N(R¹⁶)C(O)—, —C(O)N(R¹⁶)—, —N(R¹⁶)C(O)N(R¹⁶)—, —S—, —S(O)—, —S(O)₂—, —S(O)₂N(R¹⁶)—, and —N(R¹⁶)S(O)₂—;
and wherein each R¹² and each R¹⁶ are independently selected from the group consisting of hydrogen, C₁-C₅ alkyl, C₂-C₅ fluoroalkyl, C₃-C₆ cycloalkyl and heteroaryl, provided that when Z is selected from —S—, —S(O)— or —S(O)₂—, R¹² is not hydrogen;
   or R¹⁰ is C₁-C₆ alkylene-OR¹³;
wherein R¹³ is selected from the group consisting of hydrogen, C₃-C₅ alkyl, hydroxy-C₁-C₅ alkyl, C₂-C₅ fluoroalkyl, C₃-C₆ cycloalkyl and benzyl;
   or R¹⁰ is C₂-C₅ fluoroalkyl, C₄-C₆ heterocyclylalkyl or C₃-C₉ heterocyclyl, provided that said heterocyclyl is bonded via a ring carbon atom;
   y is 0, 1, 2 or 3; and
   R¹¹ is selected from the group consisting of C₁-C₄ alkyl, C₂-C₄ alkenyl, C₃-C₆ cycloalkyl, C₃-C₈ heterocyclyl, aryl, C₁-C₉ heteroaryl, C₁-C₄ fluoroalkyl, C₁-C₄ alkoxy, C₁-C₄ hydroxyalkyl, C₁-C₄ alkylthio, trifluoromethoxy, trifluoromethylthio, benzyloxy, halo, nitro, hydroxy, —OC(O)R¹⁴, —C(O)R¹⁴, —C(O)OR¹⁴, —C(O)N(R¹⁴)₂, —N(R¹⁴)₂, —N(R¹⁴)C(O)R¹⁴, —N(R¹⁴)S(O)₂R¹⁴, —S(O)₂N(R¹⁴)₂, —S(O)R¹⁴ and —S(O)₂R¹⁴;
wherein each R¹⁴ is independently selected from the group consisting of hydrogen, C₁-C₄ alkyl, C₂-C₄ alkenyl, C₂-C₄ fluoroalkyl, C₃-C₆ cycloalkyl, aryl, C₁-C₉ heteroaryl, and C₃-C₈ heterocyclyl, provided that said heterocyclyl is bonded via a ring carbon atom; and provided that when R¹¹ is selected from —S(O)R¹⁴ or —S(O)₂R¹⁴, R¹⁴ is not hydrogen;
   or two substituents R¹¹ together form a saturated or unsaturated, aliphatic or heterocyclic ring;
   and provided that the said compound is not selected from the group consisting of:
5,7-Dimethyl-6-(4-methylbenzyl)-N-(tetrahydrofuran-2-ylmethyl)-pyrazolo[1,5-a]pyrimidine-3-carboxamide;
5,7-Dimethyl-6-(3-methylbenzyl)-N-(tetrahydrofuran-2-ylmethyl)-pyrazolo[1,5-a]pyrimidine-3-carboxamide;
6-(3-Bromobenzyl)-5,7-dimethyl-N-(tetrahydrofuran-2-ylmethyl)-pyrazolo[1,5-a]pyrimidine-3-carboxamide;
5,7-Dimethyl-6-(2-methylbenzyl)-N-(tetrahydrofuran-2-ylmethyl)-pyrazolo[1,5-a]pyrimidine-3-carboxamide;
6-(2,5-Dimethylbenzyl)-5,7-dimethyl-N-(tetrahydrofuran-2-ylmethyl)-pyrazolo[1,5-a]pyrimidine-3-carboxamide;
6-(4-Fluorobenzyl)-5,7-dimethyl-N-(tetrahydrofuran-2-ylmethyl)pyrazolo[1,5-a]pyrimidine-3-carboxamide;
6-(2-Chlorobenzyl)-5,7-dimethyl-N-(tetrahydrofuran-2-ylmethyl)-pyrazolo[1,5-a]pyrimidine-3-carboxamide;
6-(2-Chloro-4-fluorobenzyl)-5,7-dimethyl-N-(tetrahydrofuran-2-ylmethyl)-pyrazolo[1,5-a]pyrimidine-3-carboxamide;
6-(2-Chloro-6-fluorobenzyl)-5,7-dimethyl-N-(tetrahydrofuran-2-ylmethyl)-pyrazolo[1,5-a]pyrimidine-3-carboxamide; and
6-(3-Fluorobenzyl)-5,7-dimethyl-N-(tetrahydrofuran-2-ylmethyl)pyrazolo[1,5-a]pyrimidine-3-carboxamide.

Preferred compounds of the formula III according to the invention are those selected from the group consisting of:
N-[2-(Acetylamino)ethyl]-5,7-dimethyl-6-(2-methylbenzyl)pyrazolo[1,5-a]-pyrimidine-3-carboxamide;
N-(3-Isopropoxypropyl)-5,7-dimethyl-6-(2-methylbenzyl)pyrazolo[1,5-a]-pyrimidine-3-carboxamide;
N-(2-Amino-2-oxoethyl)-5,7-dimethyl-6-(2-methylbenzyl)pyrazolo[1,5-a]-pyrimidine-3-carboxamide;
6-(3-Chlorobenzyl)-5,7-dimethyl-N-(tetrahydrofuran-2-ylmethyl)pyrazolo-[1,5-a]pyrimidine-3-carboxamide;
6-(3-Chlorobenzyl)-5,7-dimethyl-N-[2-(methylthio)ethyl]pyrazolo[1,5-a]-pyrimidine-3-carboxamide;
N-[2-(Acetylamino)ethyl]-6-(3-methoxybenzyl)-5,7-dimethylpyrazolo[1,5-a]-pyrimidine-3-carboxamide;
N-(3-Isopropoxypropyl)-6-(3-methoxybenzyl)-5,7-dimethylpyrazolo[1,5-a]-pyrimidine-3-carboxamide;
6-(3-Methoxybenzyl)-5,7-dimethyl-N-(tetrahydrofuran-2-ylmethyl)pyrazolo-[1,5-a]pyrimidine-3-carboxamide;
N-[2-(Acetylamino)ethyl]-6-(3-fluorobenzyl)-5,7-dimethylpyrazolo[1,5-a]-pyrimidine-3-carboxamide;
6-(3-Fluorobenzyl)-N-(3-isopropoxypropyl)-5,7-dimethylpyrazolo[1,5-a]-pyrimidine-3-carboxamide;
N-[2-(1,3-Dioxolan-2-yl)ethyl]-6-(3-fluorobenzyl)-5,7-dimethylpyrazolo[1,5-a]pyrimidine-3-carboxamide;
N-[2-(Acetylamino)ethyl]-6-benzyl-5,7-dimethylpyrazolo[1,5-a]pyrimidine-3-carboxamide;
6-Benzyl-N-(3-isopropoxypropyl)-5,7-dimethylpyrazolo[1,5-a]pyrimidine-3-carboxamide;
6-(3-Bromobenzyl)-N-(3-isopropoxypropyl)-5,7-dimethylpyrazolo[1,5-a]-pyrimidine-3-carboxamide;
N-[2-(Acetylamino)ethyl]-6-(3-bromobenzyl)-5,7-dimethylpyrazolo[1,5-a]-pyrimidine-3-carboxamide;
N-(2-Amino-2-oxoethyl)-6-(3-bromobenzyl)-5,7-dimethylpyrazolo[1,5-a]-pyrimidine-3-carboxamide;
6-(3-Bromobenzyl)-N-(2-hydroxyethyl)-5,7-dimethylpyrazolo[1,5-a]-pyrimidine-3-carboxamide;
6-(3-Bromobenzyl)-N-(2-tert-butoxyethyl)-5,7-dimethylpyrazolo[1,5-a]-pyrimidine-3-carboxamide;
6-(3-Bromobenzyl)-N-(2-isopropoxyethyl)-5,7-dimethylpyrazolo[1,5-a]-pyrimidine-3-carboxamide;
6-Benzyl-5,7-dimethyl-N-(tetrahydrofuran-2-ylmethyl)pyrazolo[1,5-a]-pyrimidine-3-carboxamide;
6-(3,4-dichlorobenzyl)-N-[2-(2-hydroxyethoxy)ethyl]-5,7-dimethylpyrazolo-[1,5-a]pyrimidine-3-carboxamide;
5,7-dimethyl-N-[3-(methylamino)-3-oxopropyl]-6-[3-(trifluoromethoxy)-benzyl]pyrazolo[1,5-a]pyrimidine-3-carboxamide;
N-{3-[(2-hydroxyethyl)amino]-3-oxopropyl}-5,7-dimethyl-6-[3-(trifluoro-methoxy)benzyl]pyrazolo[1,5-a]pyrimidine-3-carboxamide;
N-(3-amino-3-oxopropyl)-6-(3-chloro-4-fluorobenzyl)-5,7-dimethylpyrazolo-[1,5-a]pyrimidine-3-carboxamide;
N-(2-amino-2-oxoethyl)-5,7-dimethyl-6-[3-(trifluoromethyl)benzyl]pyrazolo-[1,5-a]pyrimidine-3-carboxamide;
N-(3-hydroxypropyl)-5,7-dimethyl-6-[3-(trifluoromethyl)benzyl]pyrazolo-[1,5-a]pyrimidine-3-carboxamide;
N-[2-(acetylamino)ethyl]-6-[3-(benzyloxy)benzyl]-5,7-dimethylpyrazolo[1,5-a]pyrimidine-3-carboxamide;
5,7-dimethyl-6-(3-methylbenzyl)-N-{2-[(pyrazin-2-ylcarbonyl)amino]ethyl}-pyrazolo[1,5-a]pyrimidine-3-carboxamide;
N-[2-(benzyloxy)ethyl]-5,7-dimethyl-6-[3-(trifluoromethoxy)benzyl]pyrazolo-[1,5-a]pyrimidine-3-carboxamide;
N-(3-amino-3-oxopropyl)-6-(3,5-dichlorobenzyl)-5,7-dimethylpyrazolo[1,5-a]pyrimidine-3-carboxamide;
5,7-dimethyl-N-{2-[(pyridin-3-ylcarbonyl)amino]ethyl}-6-[3-(trifluoro-methoxy)benzyl]pyrazolo[1,5-a]pyrimidine-3-carboxamide;
6-[4-fluoro-3-(trifluoromethoxy)benzyl]-N-[2-(2-hydroxyethoxy)ethyl]-5,7-dimethylpyrazolo[1,5-a]pyrimidine-3-carboxamide; and
6-[4-fluoro-3-(trifluoromethyl)benzyl]-N-[2-(2-hydroxyethoxy)ethyl]-5,7-dimethylpyrazolo[1,5-a]pyrimidine-3-carboxamide.

The compounds herein are useful as modulators of stearoyl-CoA desaturase activity and as modulators of lipid composition and levels. They are preferably useful as modulators of human stearoyl-CoA desaturase activity and as modulators of lipid composition and levels. In particular, they are useful in the treatment or prevention of cardiovascular diseases, obesity, non-insulin-dependent diabetes mellitus, hypertension, neurological diseases, immune disorders, cancer, essential fatty acid deficiency, acne, psoriasis, rosacea, or in the treatment of excessive hair growth. The invention thus includes methods for treatment or prevention of the above-mentioned conditions, comprising administering to a mammal in need of such treatment an effective amount of a compound as defined above.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabelling/assay techniques, blotting/chemiluminescence methods, real-time PCR, and the like.

In one aspect, the mammal to be treated according to the method of the present invention is man. In another aspect, the mammal to be treated according to the method of the present invention is any other mammal. Non-limiting examples of other mammals include horses, cows, sheep, goats, dogs, cats, guinea pigs, rats and other equine, bovine, ovine, canine, feline and rodent species.

The invention also includes the use of said compounds in the manufacture of a medicament for the treatment or prevention of cardiovascular diseases, obesity, non-insulin-dependent diabetes mellitus, hypertension, neurological diseases, immune disorders, cancer, essential fatty acid deficiency, acne, psoriasis, rosacea, or for the treatment of excessive hair growth.

Another aspect of the invention is a compound of the formulae herein for use in the treatment or prevention in a subject of cardiovascular diseases, obesity, non-insulin-dependent diabetes mellitus, hypertension, neurological diseases, immune disorders, cancer, essential fatty acid deficiency, acne, psoriasis, rosacea or for use in the treatment of excessive hair growth.

### DEFINITIONS

The various terms used, separately and in combinations, in the above definition of the compounds having the formula (I-III) will be explained.

Certain chemical groups named herein are preceded by a shorthand notation indicating the total number of carbon atoms that are to be found in the indicated chemical group. For example; C₁-C₁₀ alkyl denotes an alkyl group having a total of one to ten carbon atoms. The total number of carbon atoms in the shorthand notation does not include carbons that may exist in substituents of the group described.

Accordingly, as used in the specification and appended claims, unless specified to the contrary, the following terms have the meaning indicated:

“Alkyl” denotes a straight or branched hydrocarbon chain radical consisting only of carbon and hydrogen atoms, containing no unsaturation, and which is attached to the rest of the molecule by a single bond, e.g., methyl, ethyl, n-propyl, iso-propyl, n-butyl, n-pentyl, t-butyl, n-hexyl, and the like. When referring to e.g. a “C₁-C₆ alkyl” radical, all subgroups thereof are contemplated, such as C₁-C₅ alkyl, C₁-C₄ alkyl, C₁-C₃ alkyl, C₁-C₂ alkyl, C₂-C₆ alkyl, C₂-C₅ alkyl, C₂-C₄ alkyl, C₂-C₃ alkyl, C₃-C₆ alkyl, C₄-C₅ alkyl, etc.

“C₁-C₄alkyl” refers to an alkyl radical as defined above containing one to four carbon atoms.

“Alkenyl” denotes a straight or branched hydrocarbon chain radical consisting only of carbon and hydrogen atoms, containing at least one double bond, and which is attached to the rest of the molecule by a single bond, e.g., ethenyl, prop-1-enyl, but-1-enyl, pent-2-enyl, and the like. When referring to e.g. a “C₂-C₆ alkenyl” radical, all subgroups thereof are contemplated, such as C₂-C₅ alkenyl, C₂-C₄ alkenyl, C₂-C₃ alkenyl, C₁-C₂ alkenyl, C₂-C₆ alkenyl, C₃-C₄ alkenyl, C₃-C₅ alkenyl, C₄-C₅ alkenyl, C₄-C₆ alkenyl, etc.

“C₂-C₄ alkenyl” refers to an alkenyl radical as defined above containing two to four carbon atoms.

“Alkynyl” denotes a straight or branched hydrocarbon chain radical consisting only of carbon and hydrogen atoms, containing at least one triple bond, and which is attached to the rest of the molecule by a single bond, e.g., ethynyl, prop-2-ynyl, but-2-ynyl, pent-3-ynyl, and the like. When referring to e.g. a “C₃-C₆ alkynyl” radical; all subgroups thereof are contemplated, such as C₃-C₄ alkynyl, C₃-C₅ alkynyl, C₃-C₅ alkynyl, C₃-C₆ alkynyl, C₄-C₅ alkynyl, C₄-C₆ alkynyl, C₅-C₆ alkynyl, etc.

“C₃-C₄ alkenyl” refers to an alkynyl radical as defined above containing three to four carbon atoms.

“Alkylene” denotes a straight or branched divalent saturated hydrocarbon chain, linking the rest of the molecule to a radical group, consisting only of carbon and hydrogen atoms. Examples of an alkylene radical include methylene, ethylene, 1,3-propylene, 1,4-butylene, and the like. The alkylene chain may be attached to the rest of the molecule and to the radical group through one carbon within the chain or through any two carbons within the chain. When referring to e.g. a “C₁-C₄ alkylene” radical, all subgroups thereof are contemplated, such as C₁-C₃ alkylene, C₁-C₂ alkylene, C₂-C₃ alkylene, C₂-C₄ alkylene, C₃-C₄ alkylene, etc.

“C₁-C₆ alkylene” refers to an alkylene radical as defined above containing one to six carbon atoms.

“Alkylthio” denotes a radical of the formula —SRₐ where Rₐ is an alkyl radical as defined above. When referring to e.g. a “C₁-C₄ alkylhio” radical, all subgroups thereof are contemplated, such as C₁-C₃ alkylthio, C₁-C₂ alkylthio, C₂-C₃ alkylthio, C₂-C₄ alkylthio, C₃-C₄ alkylthio, etc.

“C₁-C₄ alkylthio” refers to an alkylthio radical as defined above containing one to four carbon atoms.

“Alkylthioalkyl” denotes a radical of the formula Rₐ—S—Rₐ where each Rₐ is independently an alkyl radical as defined above. The sulfur atom may be bonded to any carbon atom in either alkyl radical. When referring to e.g. a “C₁-C₆ alkylthioalkyl” radical, all subgroups thereof are contemplated, such as C₁-C₅ alkylthioalkyl, C₁-C₄ alkylthioalkyl, C₁-C₃ alkylthioalkyl, C₁-C₂ alkylthioalkyl, C₂-C₆ alkylthioalkyl, C₂-C₅ alkylthioalkyl, C₂-C₄ alkylthioalkyl, C₂-C₃ alkylthioalkyl, C₃-C₆ alkylthioalkyl, C₄-C₅ alkylthioalkyl, etc.

“C₃-C₈ alkylthioalkyl” refers to an alkylthioalkyl radical as defined above containing three to eight carbon atoms.

“Alkoxy” denotes a radical of the formula —ORₐ where Rₐ is an alkyl radical as defined above. Examples of alkoxy radicals include methoxy, ethoxy, iso-propoxy, n-propoxy, and the like. When referring to e.g. a “C₁-C₄ alkoxy” radical, all subgroups thereof are contemplated, such as C₁-C₃ alkoxy, C₁-C₂ alkoxy, C₂-C₃ alkoxy, C₂-C₄ alkoxy, C₃-C₄ alkoxy, etc.

“C₁-C₄ alkoxy” refers to an alkoxy radical as defined above containing one to four carbon atoms.

“Alkoxyalkyl” denotes a radical of the formula —Rₐ—O—Rₐ where each Rₐ is independently an alkyl radical as defined above. The oxygen atom may be bonded to any carbon atom in either alkyl radical. When referring to e.g. a “C₁-C₆ alkoxyalkyl” radical, all subgroups thereof are contemplated, such as C₁-C₅ alkoxyalkyl, C₁-C₄ alkoxyalkyl, C₁-C₃ alkoxyalkyl, C₁-C₂ alkoxyalkyl, C₂-C₆ alkoxyalkyl, C₂-C₅ alkoxyalkyl, C₂-C₄ alkoxyalkyl, C₂-C₃ alkoxyalkyl, C₃-C₆ alkoxyalkyl, C₄-C₅ alkoxyalkyl, etc.

“C₃-C₈ alkoxyalkyl” refers to an alkoxyalkyl radical as defined above containing three to eight carbon atoms.

“Aryl” denotes an aromatic monocyclic or multicyclic hydrocarbon ring system consisting only of carbon and hydrogen atoms and containing from 6 to 19 carbon atoms, preferably 6 to 10 carbon atoms, where the ring system may be partially or fully saturated but has at least one aromatic ring in the ring system. Aryl groups include, but are not limited to groups such as phenyl, naphthyl, fluorenyl, and indanyl (i.e., 2,3-dihydroindenyl). Unless otherwise stated specifically in the specification, the term “aryl” or the prefix “aryl-” (such as in “arylalkyl”) is meant to include aryl radicals that are optionally substituted by one or more substituents such as halo, hydroxy, nitro, alkyl, alkenyl, alkoxy, alkylthio, hydroxyalkyl, fluoroalkyl, trifluoromethoxy, trifluoromethylthio, cycloalkyl, heterocyclyl, aryl, heteroaryl, and aryloxy, or by two substituents that together form a saturated or unsaturated heterocyclic ring.

“Arylalkyl” denotes a radical of the formula -RₐR_{b} where Rₐ is an alkyl radical as defined above and R_{b} is one or more aryl radicals as defined above, e.g. benzyl, diphenylmethyl and the like. The aryl part of the arylalkyl radical may be optionally substituted as defined above for an aryl group. When referring to e.g. a “C₇-C₁₂ arylalkyl” radical, all subgroups thereof are contemplated, such as C₇-C₁₁ arylalkyl, C₇-C₁₀ arylalkyl, C₇-C₈ arylalkyl, C₇-C₈ arylalkyl, C₈-C₁₂ arylalkyl, C₈-C₁₁ arylalkyl,

C₈-C₁₀ arylalkyl, C₈-C₉ arylalkyl, C₉-C₁₂ arylalkyl, C₉-C₁₁ arylalkyl, etc.

“C₇-C₁₃ arylalkyl” refers to an arylalkyl radical as defined above containing seven to thirteen carbon atoms.

“Aryloxy” denotes a radical of the formula —OR_{b} where R_{b} is an aryl group as defined above. The aryl group part of the aryloxy radical may be optionally substituted as defined above for an aryl group.

“Aryloxyalkyl” denotes a radical of the formula —Rₐ—OR_{b} where Rₐ is an alkyl radical as defined above and —OR_{b} is an aryloxy radical as defined above. When referring to e.g. a “C₇-C₁₂ aryloxyalkyl” radical, all subgroups thereof are contemplated, such as C₇-C₁₁aryloxyalkyl, C₇-C₁₀ aryloxyalkyl, C₇-C₉ aryloxyalkyl, C₇-C₈ aryloxyalkyl, C₈-C₁₁ aryloxyalkyl, C₈-C₁₀ aryloxyalkyl, C₈-C₉ aryloxyalkyl, C₉-C₁₀ aryloxyalkyl, C₉-C₁₀ aryloxyalkyl, etc.

“C₈-C₁₅ aryloxyalkyl” refers to an aryloxyalkyl radical as defined above containing eight to fifteen carbon atoms.

“Cyano” refers to the —CN radical.

“Cyanoalkyl” denotes an alkyl radical, as defined above, that is substituted by a cyano radical, as defined above, e.g., cyanomethyl, 2-cyanoethyl, 3-cyanopropyl, 2-cyanobutyl, and the like. When referring to e.g. a “C₃-C₈ cyanoalkyl” radical all subgroups thereof are contemplated such as C₃-C₇ cyanoalkyl, C₃-C₆ cyanoalkyl, C₃-C₅ cyanoalkyl, C₃-C₄ cyanoalkyl, C₄-C₈ cyanoalkyl, C₄-C₇ cyanoalkyl, C₄-C₆ cyanoalkyl, C₄-C₅ cyanoalkyl, C₅-C₇ cyanoalkyl, C₆-C₇ cyanoalkyl, etc.

“C₃-C₆ cyanoalkyl” refers to a cyanoalkyl radical as defined above containing three to six carbon atoms.

“Cycloalkyl” denotes a stable non-aromatic or bicyclic hydrocarbon radical consisting only of carbon and hydrogen atoms and containing from three to fifteen carbon atoms, preferably three to ten carbon atoms, and which is saturated or unsaturated and which is attached to the rest of the molecule by a single bond, e.g., cyclopropyl, cyclobutyl, cyclopentyl, cyclohexyl, methylcyclohexyl, cycloheptyl, cyclooctyl and the like. When referring to e.g. a “C₃-C₈ cycloalkyl” radical all subgroups thereof are contemplated such as C₃-C₇ cycloalkyl, C₃-C₆ cycloalkyl, C₃-C₅ cycloalkyl, C₃-C₄ cycloalkyl, C₄-C₈ cycloalkyl, C₄-C₇ cycloalkyl, C₄-C₆ cycloalkyl, C₄-C₅ cycloalkyl, C₅-C₇ cycloalkyl, C₆-C₇ cycloalkyl, etc.

“C₃-C₆ cycloalkyl” refers to a cycloalkyl radical as defined above containing three to six carbon atoms.

“Cycloalkylalkyl” denotes a radical of the formula RₐR_{d} where Rₐ is an alkyl radical as defined above and R_{d} is a cycloalkyl radical as defined above. When referring to e.g. a “C₄-C₈ cycloalkylalkyl” radical all subgroups thereof are contemplated such as C₄-C₇ cycloalkylalkyl, C₄-C₆ cycloalkylalkyl, C₄-C₅-cycloalkylalkyl, C₅-C₈ cycloalkylalkyl, C₅-C₇ cycloalkylalkyl, C₅-C₆ cycloalkylalkyl, C₆-C₈ cycloalkylalkyl, C₆-C₇ cycloalkylalkyl, etc.

“C₄-C₁₂ cycloalkylalkyl” refers to a cycloalkylalkyl radical as defined above containing four to twelve carbon atoms.

“Halo” refers to fluoro, chloro, bromo or iodo.

“Fluoroalkyl” denotes an alkyl radical, as defined above, that is substituted by one or more fluoro radicals, e.g., trifluoromethyl, difluoromethyl, fluoromethyl, 2,2,2-trifluororoethyl, 3-fluoropropyl, 2,4-difluoropentyl, and the like. When referring to e.g. a “C₃-C₈fluoroalkyl” radical all subgroups thereof are contemplated such as C₃-C₇ fluoroalkyl, C₃-C₆ fluoroalkyl, C₃-C₅ fluoroalkyl, C₃-C₄ fluoroalkyl, C₄-C₈ fluoroalkyl, C₄-C₇ fluoroalkyl, C₄-C₆ fluoroalkyl, C₄-C₅ fluoroalkyl, C₅-C₇ fluoroalkyl, C₆-C₇ fluoroalkyl, etc.

“C₁-C₃ fluoroalkyl” refers to a fluoroalkyl radical as defined above containing one to three carbon atoms.

“Heterocyclyl” denotes a stable 3 to 18 membered non-aromatic ring radical which consists of carbon atoms and from one to five heteroatoms selected from the group consisting of nitrogen, oxygen, and sulfur. For purposes of this invention, the heterocyclyl radical may be a monocyclic, bicyclic or tricyclic ring system, which may include fused or bridged ring systems, and the nitrogen, oxygen, and sulfur atoms in the heterocyclyl radical may be optionally oxidized, and the nitrogen atom of the heterocyclyl radical may be optionally quarternized, and the heterocyclyl radical may be partially or fully saturated. Examples of such heterocyclyl radicals include, but are not limited to, dioxolanyl, decahydroisoquinolyl, imidazolinyl, imidazolidinyl, isothiazolidinyl, isoxazolidinyl, morpholinyl, octahydroindolyl, octahydroisindolyl, oxazolidinyl, piperidinyl, piperazinyl, 4-piperidonyl, pyrrolidinyl, pyrazolidinyl, thiazolidinyl, tetrahydrofuryl, tetrahydropyranyl, and thiamorpholinyl. Unless otherwise stated specifically in the specification, the term “heterocyclyl” is meant to include heterocyclyl radicals optionally substituted by one or more substituents selected from the group consisting of alkyl, alkenyl, alkoxy, halo, fluoroalkyl, cyano, oxo, thioxo, nitro, aryl and cycloalkyl. When referring to e.g. a “C₃-C₈ heterocyclyl” radical all subgroups thereof are contemplated, such as C₃-C₇ heterocyclyl, C₃-C₆ heterocyclyl, C₃-C₈ heterocyclyl, C₃-C₄ heterocyclyl, C₄-C₈ heterocyclyl, C₄-C₇ heterocyclyl, C₄-C₆ heterocyclyl, C₄-C₅ heterocyclyl, C₅-C₇ heterocyclyl, C₆-C₇ heterocyclyl, etc.

“C₃-C₈ heterocyclyl” refers to a heterocyclyl radical as defined above containing three to eight carbon atoms.

“Heterocyclylalkyl” denotes a radical of the formula -RₐRₑ, where Rₐ is an alkyl radical as defined above and Rₑ is a heterocyclyl radical as defined above, and if the heterocyclyl is a nitrogen containing heterocyclyl, the heterocyclyl may be attached to the alkyl radical at the nitrogen atom. The heterocyclyl part of the heterocyclylalkyl radical may be optionally substituted as defined above for a heterocyclyl group. When referring to e.g. a “C₃-C₈ heterocyclylalkyl” radical all subgroups thereof are contemplated such as C₃-C₇ heterocyclylalkyl, C₃-C₆ heterocyclylalkyl, C₃-C₅ heterocyclylalkyl, C₃-C₄ heterocyclylalkyl, C₄-C₈ heterocyclylalkyl, C₄-C₇ hetero-cyclylalkyl, C₄-C₆ heterocyclylalkyl, C₄-C₅ heterocyclylalkyl, C₅-C₇ heterocyclylalkyl, C₆-C₇ heterocyclylalkyl, etc.

“C₃-C₁₀ heterocyclylalkyl” refers to a heterocyclylalkyl radical as defined above containing three to ten carbon atoms.

“Heteroaryl” denotes a stable 5- to 18 membered aromatic ring radical which consists of carbon atoms and from one to five heteroatoms selected from the group consisting of nitrogen, oxygen, and sulfur. For purposes of this invention, the heteroaryl radical may be a monocyclic, bicyclic or tricyclic ring system, which may include fused or bridged ring systems, and the nitrogen, oxygen, sulfur, and selenium atoms in the heteroaryl radical may be optionally oxidized. Examples of such heteroaryl radicals include, but are not limited to, pyrrolyl, imidazolyl, thiophenyl, furanyl, thiazolyl, isothiazolyl, thiadiazolyl, oxazolyl, isoxazolyl, oxadiazolyl, pyridinyl, pyrazinyl, pyrimidinyl, pyridazinyl, pyrazolyl, triazolyl, tetrazolyl, chromanyl, isochromanyl, quinolinyl, quinoxalinyl, isoquinolinyl, phthalazinyl, quinazolinyl, indolyl, isoindolyl, benzothiophenyl, benzofuranyl, isobenzofuranyl, benzoxazolyl, 2,1,3-benzoxadiazolyl, benzopyrazolyl; benzothiazolyl, 2,1,3-benzothiazolyl, 2,1,3-benzoselenadiazolyl, benzimidazolyl, indazolyl, benzodioxinyl, 1,5-naphthyridinyl, 1,8-naphthyridinyl, pyrido[3,2-b]thiophenyl, acridinyl and fenazinyl. Unless otherwise stated specifically in the specification, the term “heteroaryl” is meant to include heteroaryl radicals optionally substituted by one or more substituents such as halo, cyano, hydroxy, oxo, thioxo, nitro, alkyl, alkenyl, alkoxy, alkylthio, hydroxyalkyl, fluoroalkyl, trifluoromethoxy, trifluoromethylthio, cycloalkyl, heterocyclyl, aryl, heteroaryl, and aryloxy. When referring to e.g. a “C₃-C₈ heteroaryl” radical all subgroups thereof are contemplated such as C₃-C₇ heteroaryl, C₃-C₆ heteroaryl, C₃-C₅ heteroaryl, C₃-C₄ heteroaryl, C₄-C₈ heteroaryl, C₄-C₇ heteroaryl, C₄-C₆ heteroaryl, C₄-C₅ heteroaryl, C₅-C₇ heteroaryl, C₆-C₇ heteroaryl, etc.

“C₁-C₉ heteroaryl” refers to a heteroaryl radical as defined above containing one to nine carbon atoms.

“Heteroarylalkyl” denotes a radical of the formula -RₐR_{f} where Rₐ is an alkyl radical as defined above and R_{f} is a heteroaryl radical as defined above. The aryl part of the heteroarylalkyl radical may be optionally substituted as defined above for a heteroaryl group. When referring to e.g. a “C₃-C₈ heteroarylalkyl” radical all subgroups thereof are contemplated, such as C₃-C₇ heteroarylalkyl, C₃-C₆ heteroarylalkyl, C₃-C₅ heteroarylalkyl, C₃-C₄ heteroarylalkyl, C₄-C₈ heteroarylalkyl, C₄-C₇ heteroarylalkyl, C₄-C₆ heteroarylalkyl, C₄-C₅ heteroarylalkyl, C₅-C₇ heteroarylalkyl, C₆-C₇ heteroarylalkyl, etc.

“C₃-C₁₂ heteroarylalkyl” refers to a heteroarylalkyl radical as defined above containing three to twelve carbon atoms.

“Hydroxy” refers to the —OH radical.

“Hydroxyalkyl” denotes a radical of the formula —Rₐ—OH where Rₐ is an alkyl radical as defined above. The hydroxy group may be attached to the alkyl radical on any carbon within the alkyl radical. When referring to e.g. a “C₃-C₈ hydroxyalkyl” radical all subgroups thereof are contemplated, such as C₃-C₇ hydroxyalkyl, C₃-C₆ hydroxyalkyl, C₃-C₅ hydroxyalkyl, C₃-C₄ hydroxyalkyl, C₄-C₈ hydroxyalkyl, C₄-C₇ hydroxyalkyl, C₄-C₆ hydroxyalkyl, C₄-C₅ hydroxyalkyl, C₅-C₇ hydroxyalkyl, C₆-C₇ hydroxyalkyl, etc.

“C₁-C₄ hydroxyalkyl” refers to a hydroxyalkyl radical as defined above containing one to four carbon atoms.

“Nitro” refers to the —NO₂ radical.

“Oxo” refers to the ═O substituent.

“Thioxo” refers to the ═S substituent.

“Prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2^{nd} Ed., (2004), pp. 498-549, Elsevier Academic Press). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

“Stereoisomer” refers to a compound made up of exactly the same atoms bonded by the same bonds, but having different three-dimensional structures, which are not interchangeable. The present invention includes various stereoisomers and mixtures thereof and includes “enantiomers”, which refers to two stereoisomers which are nonsuperimposable mirror images of one another.

“Tautomer” refers to a shift of a proton from one atom in a molecule to another atom in the same molecule. The present invention includes tautomers of any said compounds.

The chemical naming protocol used herein employ and rely on the chemical naming features of ACD/ChemSketch product version 9.08 (available from Advanced Chemical Development, Toronto, Ontario, Canada).

All isomeric forms possible (pure enantiomers, diastereomers, tautomers, racemic mixtures and unequal mixtures of two enantiomers) for the compounds delineated are within the scope of the invention. When the compounds described herein contain olefinic double bonds of geometric asymmetry, it is intended to include both trans and cis (E and Z) geometric isomers.

The compounds of the formulae herein may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### Compositions

It will be appreciated that compounds of the invention may be administered together with a physiologically acceptable carrier, excipient, or diluent. The pharmaceutical compositions of the invention include those suitable for oral, rectal, nasal, topical (including buccal and sublingual), sublingual, transdermal, intrathecal, transmucosal, vaginal or parenteral (including subcutaneous, intramuscular, intravenous and intradermal) administration. In certain embodiments, the compound of the formulae herein is administered transdermally (e.g., using a transdermal patch or iontophoretic techniques For the treatment of skin diseases, they can also be administered topically. The amount of drug administered will typically be higher when administered orally than when administered, say, intravenously.

Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, ointments, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the invention may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

The compounds of formulae herein may be administered with other active compounds for the treatment of treatment of medical conditions in which the modulation of SCD activity is beneficial, such as cardiovascular diseases, obesity, non-insulin-dependent diabetes mellitus, hypertension, neurological diseases, immune disorders, and cancer; including e.g., type 2 diabetes, coronary artery disease, atherosclerosis, heart disease, cerebrovascular disease, eczema, acne and psoriasis. Such agents are known in the art and include those delineated in the references cited herein, as well as, e.g., insulin and insulin analogs, DPP-IV inhibitors, sulfonyl ureas, biguanides, α2 agonists, glitazones, PPAR-γ agonists, mixed PPAR-α/γ agonists, RXR agonists, α-glucosidase inhibitors, PTP1B inhibitors, 11-β-hydroxy steroid dehydrogenase Type 1 inhibitors, phosphodiesterase inhibitors, glycogen phosphorylase inhibitors, MCH-I antagonists, CB-I antagonists (or inverse agonists), amylin antagonists, CCK receptor agonists, β₃-agonists, leptin and leptin mimetics, serotonergic/dopaminergic antiobesity drugs, gastric lipase inhibitors, pancreatic lipase inhibitors, fatty acid oxidation inhibitors, lipid lowering agents and thyromimetics.

### Preparation of Compounds of the Invention

It is well known by those skilled in the art that in the process described below, functional groups, such as amino, hydroxy and carboxylic acid of intermediates may have to be protected by suitable protecting groups. For the amino group suitable protecting groups may include trifluoroacetamide, tert-butoxycarbonyl, 9-fluorenylmethoxycarbonyl, benzyloxycarbonyl and the like. Suitable protecting groups for the hydroxy group include trimethylsilyl, tert-butyldimethylsilyl, p-methoxybenzyl, benzyloxymethyl, tetrahydropyranyl and the like. Suitable groups for the carboxylic acid group include methyl esters, tert-butyl esters, p-nitrobenzyl esters, allyl esters and the like. The protective groups are added to and removed from the intermediate compound according to standard protocols, which are well known to those skilled in the art.

Protective group chemistry is described in detail in Kocieñski, P. J., Protecting Groups (2000), Corrected Edition, Georg Thieme Verlag, Stuttgart.

In the following Reaction Schemes methods to make compounds of the invention are described. The compounds described herein would be possible to make by those skilled in the art by similar or other methods, described elsewhere or known by those skilled in the art. Compounds used as starting material may be obtained from commercial sources such as Sigma Aldrich, Maybridge, Matrix Scientific, Lancaster Synthesis, Avocado Organics, ASDI etc. They may also be synthesized by methods known by those skilled in the art or according to methods described elsewhere.

In general the compounds of Formula I of this invention can be synthesized by the general procedure described in Reaction Scheme 1, where x=0 and W=—C(O)N(R⁶)—. Definitions of variables in the structures in schemes herein are commensurate with those of corresponding positions in the formulae delineated herein.

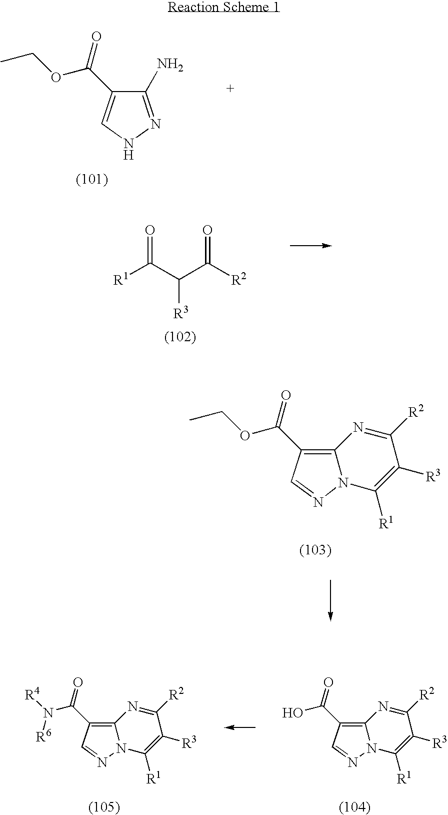

The starting materials for the synthesis of compounds made according to the above reaction scheme are commercially available or can be synthesized by methods known by those skilled in the art or by methods disclosed herein or elsewhere.

The aminopyrazole 101 is reacted with the 1,3-dicarbonyl compound 102 in the presence of an acid such as, but not limited to, hydrochloric acid in a refluxing solvent such as, but not limited to, ethanol to form the product 103. Hydrolysis of the ester group of 103 to form the carboxylic acid 104 can be achieved by using a base such as, but not limited to, potassium hydroxide. Conversion of the carboxylic acid group of 104 to the corresponding amide 105 can be performed by reaction with the appropriate amine in the presence of a coupling reagent such as, but not limited to, 1-propanephosphonic acid cyclic anhydride at room temperature in a solvent such as, but not limited to, N,N-dimethylformamide.

Even though anyone skilled in the art is capable of preparing the compounds of the invention according to the general scheme disclosed above, more specific details for the synthesis of compounds of the invention are provided elsewhere in this specification for convenience.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

### Intermediate 1

### Synthesis of 3-(4-chlorobenzyl)pentane-2,4-dione

A mixture of pentane-2,4-dione (2.402 g, 24.0 mmol) and p-chlorobenzyl bromide (2.466 g, 12.0 mmol) in toluene (12 mL) were heated in a microwave reactor at 170° C. for 45 min. After cooling to room temperature, water (20 mL) and diethyl ether (160 mL) were added and the phases were separated. The organic phase was washed with an additional 2×20 mL of water and brine (20 mL). The organic phase was dried over MgSO₄ and concentrated in vacuo to afford 2,589 g (96%) of the title product. According to HPLC-MS and ¹H NMR the product was essentially pure (91%) and a mixture of keto and enol forms. The material was taken to the next step without further purification. MS (ESI) m/z 225 [M+H⁺].

### Intermediate 2

### Synthesis of 3-(3-methoxybenzyl)pentane-2,4-dione

To a solution of pentane-2,4-dione (1.00 g, 10 mmol) in tetrahydrofuran (2.5 mL) tetrabutylammonium fluoride in tetrahydrofuran (1M, 10 mL) was added followed by water (1 mL). The mixture was stirred at room temperature for 10 min and then 3-methoxybenzyl bromide (2.01 g, 10 mmol) was added. After stirring for 25 h at room temperature the mixture was concentrated and the residue was purified by flash chromatography (silica, 10-30% ethyl acetate in n-hexane) to afford 0.902 g (41%) of the title product as colorless oil. MS (ESI) m/z 221 [M+H⁺].

### Intermediate 3

### Synthesis of ethyl 6-(4-chlorobenzyl)-5,7-dimethylpyrazolo[1,5-a]pyrimidine-3-carboxylate

Hydrochloric acid (7 mL of a 2M solution in diethyl ether) was added to a mixture of 3-(4-chlorobenzyl)pentane-2,4-dione (2,589 g, 11.5 mmol) and ethyl 3-amino-1H-pyrazole-4-carboxylate (1.784 g, 11.5 mmol) in ethanol (36 mL). The mixture was heated to reflux for 16 h and then the reaction mixture was concentrated and the residue was treated with diethyl ether to produce a light gray precipitate. The solid was filtered off and dried to yield 3.643 g (85%) of crude title product as the corresponding hydrochloride salt. The product was used in the next step without further purification. MS (ESI) m/z 344 [M+H⁺].

### Intermediate 4

### Synthesis of 6-(4-chlorobenzyl)-5,7-dimethylpyrazolo[1,5-a]pyrimidine-3-carboxylic Acid

Potassium hydroxide (12 mL of a 2M aqueous solution, 24.0 mmol) was added to a slurry of 6-(4-chlorobenzyl)-5,7-dimethylpyrazolo[1,5-a]pyrimidine-3-carboxylic acid (3.438 g, 10.0 mmol) in methanol (25 mL) and the mixture was refluxed for 2.5 h. After cooling to room temperature water (120 mL) was added and the aqueous phase was washed with chloroform (2×50 mL). The aqueous phase was concentrated somewhat in order to remove possible remaining chloroform and was then acidified to pH 5 by addition of concentrated sulfuric acid which resulted in the formation of white precipitate. The solid was filtered off and washed with a mixture of water and methanol and dried to yield 2,590 g (82%) of the title product as white solid. MS (ESI) m/z 316 [M+H⁺].

### Intermediate 5

### Synthesis of 3-(3-chlorobenzyl)pentane-2,4-dione

A mixture of pentane-2,4-dione (2.00 g, 10.0 mmol), 3-chlorobenzyl bromide (2.05 g, 10.0 mmol) and Li₂CO₃ (1.48 g, 20.0 mmol) was heated in DMF (30 mL) at 75° C. for 1 h. The mixture was then poured on sat NaCl and acidified with sat HCl. Toluene (100 mL) was used to extract the product. The organic phase was washed twice with sat NaCl, dried (Na₂SO₄), filtered and evaporated to 2.05 g (91%) clear oil, pure as a tautomeric mixture. MS (ESI) m/z 225 [M+H⁺].

### Intermediate 6

### Synthesis of 6-(3-chlorobenzyl)-2,5,7-trimethylpyrazolo[1,5-a]pyrimidine-3-carboxylic Acid

Ethyl 5-amino-3-methyl-1H-pyrazole-4-carboxylate (207 mg, 1.2 mmol) and 3-(3-chlorobenzyl)pentane-2,4-dione (Intermediate 5) (250 mg, 1.1 mmol) were dissolved in ethanol (5 mL) and acidified with a catalytic amount of sat HCl. The reaction mixture was heated at 75° C. for 30 min to afford the cyclization. 1 M KOH (5 mL) was then added and the mixture heated at 75° C. overnight. The reaction mixture was then cooled and 1 M HCl added until pH<1, which precipitated the acid. Filtering, washing with 1 M HCl and drying gave 203 mg (56%) off-white solid 6-(3-chlorobenzyl)-2,5,7-trimethylpyrazolo[1,5-a]pyrimidine-3-carboxylic acid. MS (ESI) m/z 331 [M+H⁺].

### Intermediate 7

### Synthesis of 3-(3-chloro-4-fluorobenzyl)pentane-2,4-dione

A mixture of pentane-2,4-dione (2.00 g, 10.0 mmol), 4-(bromomethyl)-2-chloro-1-fluorobenzene (2.23 g, 10.0 mmol) and Li₂CO₃ (1.48 g, 20.0 mmol) was heated in DMF (30 mL) at 75° C. for 1 h. The mixture was then poured on sat NaCl and acidified with sat HCl. Toluene (100 mL) was used to extract the product. The organic phase was washed twice with sat NaCl, dried (Na₂SO₄), filtered and evaporated to 2.16 g (92%) clear oil, pure as a tautomeric mixture. MS (ESI) m/z 236 [M+H⁺].

### Intermediate 8

### Synthesis of 6-(3,4-dichlorobenzyl)-5,7-dimethylpyrazolo[1,5-a]pyrimidine-3-carboxylic Acid

Following the procedure described to synthesize Intermediate 5-6, but using 3,4-dichlorobenzyl bromide in 2.0 mmol scale, gave 491 mg off-white solid (70%) of the title intermediate.

### Intermediate 9

### Synthesis of 6-(3-chloro-4-fluorobenzyl)-5,7-dimethylpyrazolo[1,5-a]pyrimidine-3-carboxylic Acid

Ethyl 5-amino-1H-pyrazole-4-carboxylate (186 mg, 1.2 mmol) and 3-(3-chloro-4-fluorobenzyl)pentane-2,4-dione (Intermediate 7) (267 mg, 1.1 mmol) were subjected to the synthetic procedure used to produce Intermediate 6, yielding 235 mg (64%) off-white solid. MS (ESI) m/z 334 [M+H⁺].

### Intermediate 10

### Synthesis of 5,7-dimethyl-6-[3-(trifluoromethyl)benzyl]pyrazolo[1,5-a]pyrimidine-3-carboxylic Acid

Following the procedure described to synthesize Intermediate 5-6, but using 1-(bromomethyl)-3-(trifluoromethyl)benzene in 10 mmol scale, gave 2.82 g white solid ester (79%), which was hydrolyzed to the title intermediate.

### Intermediate 11

### Synthesis of 6-(3,5-dichlorobenzyl)-5,7-dimethylpyrazolo[1,5-a]pyrimidine-3-carboxylic Acid

Following the procedure described to synthesize Intermediate 5-6, but using 3,5-dichlorobenzyl chloride in 2.0 mmol scale, gave 114 mg off-white solid (16%) of the title intermediate.

### Intermediate 12

### Synthesis of 5,7-dimethyl-6-[3-(trifluoromethoxy)benzyl]pyrazolo[1,5-a]pyrimidine-3-carboxylic Acid

Following the procedure described to synthesize Intermediate 5-6, but using 1-(bromomethyl)-3-(trifluoromethoxy)benzene in 2.0 mmol scale, gave 333 mg off-white solid (46%) of the title intermediate.

### Intermediate 13

### Synthesis of 6-(2,5-dichlorobenzyl)-5,7-dimethylpyrazolo[1,5-a]pyrimidine-3-carboxylic Acid

Following the procedure described to synthesize Intermediate 5-6, but using 2-(bromomethyl)-1,4-dichlorobenzene in 2.0 mmol scale, gave 471 mg off-white solid (67%) of the title intermediate.

### Intermediate 14

### Synthesis of 6-(4-bromobenzyl)-5,7-dimethylpyrazolo[1,5-a]pyrimidine-3-carboxylic Acid

Following the procedure described to synthesize Intermediate 5-6, but using 1-bromo-4-(bromomethyl)benzene in 2.0 mmol scale, gave 488 mg off-white solid (68%) of the title intermediate.

### Intermediate 15

### Synthesis of 6-[3-(benzyloxy)benzyl]-5,7-dimethylpyrazolo[1,5-a]pyrimidine-3-carboxylic Acid

Following the procedure described to synthesize Intermediate 5-6, but using 1-(benzyloxy)-3-(bromomethyl)benzene in 2.0 mmol scale, gave 682 mg off-white solid (88%) of the title intermediate.

### EXAMPLES

The synthesis of compounds of this invention are illustrated by, but not limited to the following examples.

### Example 1

### Synthesis of 6-benzyl-N-(2-methoxyethyl)-5,7-dimethylpyrazolo[1,5-a]pyrimidine-3-carboxamide

A mixture of 6-benzyl-5,7-dimethylpyrazolo[1,5-a]pyrimidine-3-carboxylic acid (112 mg, 0.40 mmol), N,N-diisopropylethylamine (77 mg, 0.60 mmol) and 1-propanephosphonic acid cyclic anhydride (0.356 mL of 50% solution in ethylacetate, 0.60 mmol) in N,N-dimethylformamide (4 mL) was stirred for 10 minutes at room temperature, then 2-methoxy-ethylamine (45 mg, 0.60 mmol) was added. The reaction mixture was stirred for 1 day at room temperature. Toluene (10 mL) was added and the organic phase was washed with 0.25 M citric acid (2×4 mL), 1 M KOH (2×4 mL) and brine (4 mL). The organic phase was dried over MgSO₄ and concentrated in vacuo to yield 106 mg (78%) of the title product as white solid. MS (ESI+) calcd for C₁₉H₂₂N₄O₂ 338.1743, found 338.1746.

### Example 2

### Synthesis of 6-benzyl-N-(3-isopropoxypropyl)-5,7-dimethylpyrazolo[1,5-a]pyrimidine-3-carboxamide

To a solution of 6-benzyl-5,7-dimethylpyrazolo[1,5-a]pyrimidine-3-carboxylic acid (14 mg, 50 μmol) in N,N-dimethylformamide (200 μL) were added N,N-diisopropylethylamine (26 μL, 19 mg, 150 μmol) and 1-propanephosphonic acid cyclic anhydride (45 μL of 50% solution in ethylacetate, 24 mg, 75 μmol). The mixture was stirred for 1 h at room temperature and then 3-isopropoxypropan-1-amine (7 mg, 60 μmol) in acetonitrile (200 μL) was added. The reaction mixture was left at room temperature for one week with occasional shaking. The crude mixture was purified by reversed phase preparative HPLC to yield 8.3 mg (44%) of pure title product. MS (ESI+) calcd for C₂₂H₂₈N₄O₂ 380.2212, found 380.2223.

### Example 3

### Synthesis of N-[2-(Acetylamino)ethyl]-5,7-dimethyl-6-(2-methylbenzyl)pyrazolo-[1,5-a]pyrimidine-3-carboxamide

Following the procedure as described in Example 2, making variations only as required to use N-(2-aminoethyl)acetamide instead of 3-isopropoxypropan-1-amine and 5,7-dimethyl-6-(2-methylbenzyl)pyrazolo[1,5-a]pyrimidine-3-carboxylic acid instead of 6-benzyl-5,7-dimethylpyrazolo[1,5-a]pyrimidine-3-carboxylic acid, the title compound was obtained in 54% yield. MS (ESI+) calcd for C₂₁H₂₅N₅O₂ 379.2008, found 379.2013.

### Example 4

### Synthesis of N-(3-Isopropoxypropyl)-5,7-dimethyl-6-(2-methylbenzyl)pyrazolo[1,5-a]pyrimidine-3-carboxamide

Following the procedure as described in Example 2, making variations only as required to use 5,7-dimethyl-6-(2-methylbenzyl)pyrazolo[1,5-a]pyrimidine-3-carboxylic acid instead of 6-benzyl-5,7-dimethylpyrazolo[1,5-a]pyrimidine-3-carboxylic acid, the title compound was obtained in 58% yield. MS (ESI+) calcd for C₂₃H₃₀N₄O₂ 394.2369, found 394.2377.

### Example 5

### Synthesis of N-(2-Amino-2-oxoethyl)-5,7-dimethyl-6-(2-methylbenzyl)pyrazolo[1,5-a]pyrimidine-3-carboxamide

Following the procedure as described in Example 2, making variations only as required to use glycinamide instead of 3-isopropoxypropan-1-amine and 5,7-dimethyl-6-(2-methylbenzyl)pyrazolo[1,5-a]pyrimidine-3-carboxylic acid instead of 6-benzyl-5,7-dimethylpyrazolo[1,5-a]pyrimidine-3-carboxylic acid, the title compound was obtained in 52% yield. MS (ESI+) calcd for C₁₉H₂₁N₅O₂ 351.1695, found 351.1693.

### Example 6

### Synthesis of 6-(3-Chlorobenzyl)-5,7-dimethyl-N-(tetrahydrofuran-2-ylmethyl)-pyrazolo[1,5-a]pyrimidine-3-carboxamide

Following the procedure as described in Example 2, making variations only as required to use 1-(tetrahydrofuran-2-yl)methanamine instead of 3-isopropoxypropan-1-amine and 6-(3-chlorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 39% yield. MS (ESI+) calcd for C₂₁H₂₃ClN₄O₂ 398.151, found 398.1511.

### Example 7

### Synthesis of 6-(3-Chlorobenzyl)-5,7-dimethyl-N-[2-(methylthio)ethyl]pyrazolo[1,5-a]pyrimidine-3-carboxamide

Following the procedure as described in Example 2, making variations only as required to use 2-(methylthio)ethanamine instead of 3-isopropoxypropan-1-amine and 6-(3-chlorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 34% yield. MS (ESI+) calcd for C₁₉H₂₁ClN₄OS 388.1125, found 388.1128.

### Example 8

### Synthesis of N-[2-(Acetylamino)ethyl]-6-(3-methoxybenzyl)-5,7-dimethylpyrazolo-[1,5-a]pyrimidine-3-carboxamide

Following the procedure as described in Example 2, making variations only as required to use N-(2-aminoethyl)acetamide instead of 3-isopropoxypropan-1-amine and 6-(3-methoxybenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 63% yield. MS (ESI+) calcd for C₂₁H₂₅N₅O₃ 395.1957, found 395.1962.

### Example 9

### Synthesis of N-(3-Isopropoxypropyl)-6-(3-methoxybenzyl)-5,7-dimethylpyrazolo-[1,5-a]pyrimidine-3-carboxamide

Following the procedure as described in Example 2, making variations only as required to use 6-(3-methoxybenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 66% yield. MS (ESI+) calcd for C₂₃H₃₀N₄O₃ 410.2318, found 410.2319.

### Example 10

### Synthesis of 6-(3-Methoxybenzyl)-5,7-dimethyl-N-(tetrahydrofuran-2-ylmethyl)-pyrazolo[1,5-a]pyrimidine-3-carboxamide

Following the procedure as described in Example 2, making variations only as required to use 1-(tetrahydrofuran-2-yl)methanamine instead of 3-isopropoxypropan-1-amine and 6-(3-methoxybenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 68% yield. MS (ESI+) calcd for C₂₂H₂₆N₄O₃ 394.2005, found 394.202.

### Example 11

### Synthesis of N-[2-(Acetylamino)ethyl]-6-(3-fluorobenzyl)-5,7-dimethylpyrazolo[1,5-a]pyrimidine-3-carboxamide

Following the procedure as described in Example 2, making variations only as required to use N-(2-aminoethyl)acetamide instead of 3-isopropoxypropan-1-amine and 6-(3-fluorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 42% yield. MS (ESI+) calcd for C₂₀H₂₂FN₅O₂ 383.1758, found 383.1759.

### Example 12

### Synthesis of 6-(3-Fluorobenzyl)-N-(3-isopropoxypropyl)-5,7-dimethylpyrazolo[1,5-a]pyrimidine-3-carboxamide

Following the procedure as described in Example 2, making variations only as required to use 6-(3-fluorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 43% yield. MS (ESI+) calcd for C₂₂H₂₇FN₄O₂ 398.2118, found 398.212.

### Example 13

### Synthesis of N-[2-(1,3-Dioxolan-2-yl)ethyl]-6-(3-fluorobenzyl)-5,7-dimethyl-pyrazolo[1,5-a]pyrimidine-3-carboxamide

Following the procedure as described in Example 2, making variations only as required to use 2-(1,3-dioxolan-2-yl)ethanamine instead of 3-isopropoxypropan-1-amine and 6-(3-fluorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 45% yield. MS (ESI+) calcd for C₂₁H₂₃FN₄O₃ 398.1754, found 398.1763.

### Example 14

### Synthesis of N-[2-(Acetylamino)ethyl]-6-benzyl-5,7-dimethylpyrazolo[1,5-a]pyrimidine-3-carboxamide

Following the procedure as described in Example 2, making variations only as required to use N-(2-aminoethyl)acetamide instead of 3-isopropoxypropan-1-amine, the title compound was obtained in 39% yield. MS (ESI+) calcd for C₂₀H₂₃N₅O₂ 365.1852, found 365.1857.

### Example 15

### Synthesis of 6-Benzyl-5,7-dimethyl-N-(tetrahydrofuran-2-ylmethyl)pyrazolo[1,5-a]pyrimidine-3-carboxamide

Following the procedure as described in Example 2, making variations only as required to use 1-(tetrahydrofuran-2-yl)methanamine instead of 3-isopropoxypropan-1-amine, the title compound was obtained in 51% yield. MS (ESI+) calcd for C₂₁H₂₄N₄O₂ 364.1899, found 364.1912.

### Example 16

### Synthesis of 6-(3-Bromobenzyl)-N-(3-isopropoxypropyl)-5,7-dimethylpyrazolo[1,5-a]pyrimidine-3-carboxamide

Following the procedure as described in Example 2, making variations only as required to use 6-(3-bromobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 37% yield. MS (ESI+) calcd for C₂₂H₂₇BrN₄O₂ 458.1317, found 458.1318.

### Example 17

### Synthesis of N-[2-(Acetylamino)ethyl]-6-(3-bromobenzyl)-5,7-dimethylpyrazolo-[1,5-a]pyrimidine-3-carboxamide

Following the procedure as described in Example 2, making variations only as required to use N-(2-aminoethyl)acetamide instead of 3-isopropoxypropan-1-amine and 6-(3-bromobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 40% yield. MS (ESI+) calcd for C₂₀H₂₂BrN₅O₂ 443.0957, found 443.0954.

### Example 18

### Synthesis of N-(2-Amino-2-oxoethyl)-6-(3-bromobenzyl)-5,7-dimethylpyrazolo[1,5-a]pyrimidine-3-carboxamide

Following the procedure as described in Example 2, making variations only as required to use glycinamide instead of 3-isopropoxypropan-1-amine and 6-(3-bromobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 32% yield. MS (ESI+) calcd for C₁₈H₁₈BrN₅O₂ 415.0644, found 415.0644.

### Example 19

### Synthesis of 6-(3-Bromobenzyl)-N-(2-tert-butoxyethyl)-5,7-dimethylpyrazolo[1,5-a]pyrimidine-3-carboxamide

Following the procedure as described in Example 2, making variations only as required to use 2-tert-butoxyethanamine instead of 3-isopropoxypropan-1-amine and 6-(3-bromobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 30% yield. MS (ESI+) calcd for C₂₂H₂₇BrN₄O₂ 458.1317, found 458.1315.

### Example 20

### Synthesis of 6-(3-Bromobenzyl)-N-(2-isopropoxyethyl)-5,7-dimethylpyrazolo[1,5-a]pyrimidine-3-carboxamide

Following the procedure as described in Example 2, making variations only as required to use 2-isopropoxyethanamine instead of 3-isopropoxypropan-1-amine and 6-(3-bromobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 47% yield. MS (ESI+) calcd for C₂₁H₂₅BrN₄O₂ 444.1161, found 444.1162.

### Example 21

### Synthesis of 6-(3-chlorobenzyl)-N-(3-isopropoxypropyl)-5,7-dimethylpyrazolo[1,5-a]pyrimidine-3-carboxamide

Following the procedure as described in Example 2, making variations only as required to use 6-(3-chlorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 38% yield. MS (ESI+) calcd for C₂₂H₂₇ClN₄O₂ 414.1823, found 414.182.

### Example 22

### Synthesis of 6-(3-chlorobenzyl)-N-[2-(1,3-dioxolan-2-yl)ethyl]-5,7-dimethyl-pyrazolo[1,5-a]pyrimidine-3-carboxamide

Following the procedure as described in Example 2, making variations only as required to use 2-(1,3-dioxolan-2-yl)ethanamine instead of 3-isopropoxypropan-1-amine and 6-(3-chlorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 35% yield. MS (ESI+) calcd for C₂₁H₂₃ClN₄O₃ 414.1459, found 414.146.

### Example 23

### Synthesis of N-(2-acetamidoethyl)-6-(3-chlorobenzyl)-5,7-dimethylpyrazolo[1,5-a]-pyrimidine-3-carboxamide

Following the procedure as described in Example 2, making variations only as required to use N-(2-aminoethyl)acetamide instead of 3-isopropoxypropan-1-amine and 6-(3-chlorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 36% yield. MS (ESI+) calcd for C₂₀H₂₂ClN₅O₂ 399.1462, found 399.1461.

### Example 24

### Synthesis of N-(3-ethoxypropyl)-6-(3-fluorobenzyl)-5,7-dimethylpyrazolo[1,5-a]-pyrimidine-3-carboxamide

Following the procedure as described in Example 2, making variations only as required to use 3-ethoxypropan-1-amine instead of 3-isopropoxypropan-1-amine and 6-(3-fluorobenzyl)-5,7-dimethylpyrazolo[1,5-a]pyrimidine-3-carboxylic acid instead of 6-benzyl-5,7-dimethylpyrazolo[1,5-a]pyrimidine-3-carboxylic acid, the title compound was obtained in 49% yield. MS (ESI+) calcd for C₂₁H₂₅FN₄O₂ 384.1962, found 384.1962.

### Example 25

### Synthesis of 6-(3-Bromobenzyl)-N-(2-hydroxyethyl)-5,7-dimethylpyrazolo[1,5-a]-pyrimidine-3-carboxamide

To a solution of 6-(3-bromobenzyl)-5,7-dimethylpyrazolo[1,5-a]pyrimidine-3-carboxylic acid (18 mg, 50 μmol) in N,N-dimethylformamide (200 μL) were added N,N-diisopropylethylamine (17 μL, 12 mg, 100 μmol) and 1-propanephosphonic acid cyclic anhydride (36 μL of 50% solution in ethylacetate, 60 μmol). The mixture was stirred for 1 h at room temperature and then 2-{[tert-butyl(dimethyl)silyl]oxy}-ethanamine (13 mg, 75 μmol) in acetonitrile (200 μL) was added. The reaction mixture was left at room temperature for one week with occasional shaking. Then ammonium fluoride (28 mg, 0.75 mmol) was added and the reaction mixture was left for another 2 h until the deprotection of the t-butyldimethylsilyl group was complete. The crude mixture was purified by reversed phase preparative HPLC to yield 8.3 mg (40%) of pure title product. MS (ESI+) calcd for C₁₈H₁₉BrN₄O₂ 402.0691, found 402.0692.

### Example 26

### Synthesis of 6-(3,4-dichlorobenzyl)-N-[2-(2-hydroxyethoxy)ethyl]-5,7-dimethyl-pyrazolo[1,5-a]pyrimidine-3-carboxamide

6-(3,4-dichlorobenzyl)-5,7-dimethylpyrazolo[1,5-a]pyrimidine-3-carboxylic acid (Inter-mediate 8) (14 mg, 0.040 mmol), 2-(2-aminoethoxy)ethanol (5.0 mg, 0.048 mmol), DIPEA (16 mg, 0.120 mmol) and 1-propanephosphonic acid cyclic anhydride (0.035 mL of a 50% solution in ethylacetate, 0.062 mmol) were dissolved in dry DMF (0.5 mL) and heated at 60° C. over the weekend. The product mixture was then purified using reversed phase preparative HPLC to 11.8 mg (54%) of the title compound. MS (ESI+) calcd for C₂₀H₂₂Cl₂N₄O₃ 436.1069, found 436.1071.

### Example 27

### Synthesis of 5,7-dimethyl-N-[3-(methylamino)-3-oxopropyl]-6-[3-(trifluoro-methoxy)benzyl]pyrazolo[1,5-a]pyrimidine-3-carboxamide

5,7-dimethyl-6-[3-(trifluoromethoxy)benzyl]pyrazolo[1,5-a]pyrimidine-3-carboxylic acid (Intermediate 12) (109 mg, 0.306 mmol), methyl beta-alaninate hydrochloride (51 mg, 0.367 mmol), DIPEA (142 mg, 1.10 mmol) and 1-propanephosphonic acid cyclic anhydride (292 mg, 0.458 mmol of a 50% solution in ethylacetate) were dissolved in dry DMF (3.5 mL) and heated at 50° C. for 40 h. The reaction mixture was then diluted with 50 mL EtOAc and washed with 1 M HCl (2×25 mL) and sat Na₂CO₃ (5×25 mL). The organic layer was dried with MgSO₄ and the solvent was evaporated to 100 mg (74%) methyl N-({5,7-dimethyl-6-[3-(trifluoromethoxy)benzyl]pyrazolo[1,5-a]pyrimidin-3-yl}carbonyl)-beta-alaninate, used in next step without further purification.

Methyl N-({5,7-dimethyl-6-[3-(trifluoromethoxy)benzyl]pyrazolo[1,5-a]pyrimidin-3-yl}carbonyl)-beta-alaninate (97 mg, 0.216 mmol) from the previous step, was dissolved in THF/H₂O (1/1, 2 mL) and LiOH×H₂O (13 mg, 0.323 mmol) was added. After 3 h the reaction mixture was evaporated and purified with preparative HPLC to 85 mg (90%) N-({5,7-dimethyl-6-[3-(trifluoromethoxy)benzyl]pyrazolo[1,5-a]pyrimidin-3-yl}carbonyl)-beta-alanine, used directly in the next step. The last step was performed using the procedure in Example 26, but with N-({5,7-dimethyl-6-[3-(trifluoromethoxy)benzyl]pyrazolo[1,5-a]pyrimidin-3-yl}carbonyl)-beta-alanine and methylamine hydrochloride, resulting in 3.0 mg (15%) of the title compound. MS (ESI+) calcd for C₂₁H₂₂F₃N₅O₃ 449.1675, found 449.1687.

### Example 28

### Synthesis of N-{3-[(2-hydroxyethyl)amino]-3-oxopropyl}-5,7-dimethyl-6-[3-(trifluoromethoxy)benzyl]pyrazolo[1,5-a]pyrimidine-3-carboxamide

Following the procedure described in Example 26, but using N-({5,7-dimethyl-6-[3-(trifluoromethoxy)benzyl]pyrazolo[1,5-a]pyrimidin-3-yl}carbonyl)-beta-alanine (Intermediate in Example 27) and 2-aminoethanol, 2.7 mg (13%) of the title compound was obtained. MS (ESI+) calcd for C₂₂H₂₄F₃N₅O₄ 479.178, found 479.1782.

### Example 29

### Synthesis of 6-(3-chlorobenzyl)-N-(2-methoxyethyl)-2,5,7-trimethylpyrazolo[1,5-a]-pyrimidine-3-carboxamide

Following the procedure described in Example 26, but using 6-(3-chlorobenzyl)-2,5,7-trimethylpyrazolo[1,5-a]pyrimidine-3-carboxylic acid (Intermediate 6) and 2-methoxyethanamine, 3.1 mg (20%) of the title product was obtained. MS (ESI+) calcd for C₂₀H₂₃ClN₄O₂ 386.151, found 386.151.

### Example 30

### Synthesis of 6-(3-chloro-4-fluorobenzyl)-N-(3-methoxypropyl)-5,7-dimethyl-pyrazolo[1,5-a]pyrimidine-3-carboxamide

Following the procedure described in Example 26, but using 6-(3-chloro-4-fluorobenzyl)-5,7-dimethylpyrazolo[1,5-a]pyrimidine-3-carboxylic acid (Intermediate 9) and 3-methoxypropan-1-amine, 7.4 mg (46%) of the title compound was obtained. MS (ESI+) calcd for C₂₀H₂₂ClFN₄O₂ 404.1415, found 404.1416.

### Example 31

### Synthesis of N-(3-amino-3-oxopropyl)-6-(3-chloro-4-fluorobenzyl)-5,7-dimethyl-pyrazolo[1,5-a]pyrimidine-3-carboxamide

Following the procedure described in Example 26, but using 6-(3-chloro-4-fluorobenzyl)-5,7-dimethylpyrazolo[1,5-a]pyrimidine-3-carboxylic acid (Intermediate 9) and beta-alaninamide hydrochloride, 8.4 mg (52%) of the title compound was obtained. MS (ESI+) calcd for C₁₉H₁₉ClFN₅O₂ 403.1211, found 403.1209.

### Example 32

### Synthesis of N-(3-methoxypropyl)-5,7-dimethyl-6-[3-(trifluoromethyl)benzyl]-pyrazolo[1,5-a]pyrimidine-3-carboxamide

Following the procedure described in Example 26, but using 5,7-dimethyl-6-[3-(trifluoromethyl)benzyl]pyrazolo[1,5-a]pyrimidine-3-carboxylic acid (Intermediate 10) and 3-methoxypropan-1-amine, 4.6 mg (27%) of the title compound was obtained. MS (ESI+) calcd for C₂₁H₂₃F₃N₄O₂ 420.1773, found 420.1779.

### Example 33

### Synthesis of 5,7-dimethyl-N-[2-(1-methyl-1H-imidazol-4-yl)ethyl]-6-[3-(trifluoro-methyl)benzyl]pyrazolo[1,5-a]pyrimidine-3-carboxamide

Following the procedure described in Example 26, but using 5,7-dimethyl-6-[3-(trifluoromethyl)benzyl]pyrazolo[1,5-a]pyrimidine-3-carboxylic acid (Intermediate 10) and 2-(1-methyl-1H-imidazol-4-yl)ethanamine, 6.4 mg (35%) of the title compound was obtained. MS (ESI+) calcd for C₂₃H₂₃F₃N₆O 456.1885, found 456.1888.

### Example 34

### Synthesis of N-(2-amino-2-oxoethyl)-5,7-dimethyl-6-[3-(trifluoromethyl)benzyl]-pyrazolo[1,5-a]pyrimidine-3-carboxamide

Following the procedure described in Example 26, but using 5,7-dimethyl-6-[3-(trifluoromethyl)benzyl]pyrazolo[1,5-a]pyrimidine-3-carboxylic acid (Intermediate 10) and glycinamide hydrochloride, 7.7 mg (47%) of the title compound was obtained. MS (ESI+) calcd for C₁₉H₁₈F₃N₅O₂ 405.1413, found 405.1414.

### Example 35

### Synthesis of N-(3-hydroxypropyl)-5,7-dimethyl-6-[3-(trifluoromethyl)benzyl]-pyrazolo[1,5-a]pyrimidine-3-carboxamide

Following the procedure described in Example 26, but using 5,7-dimethyl-6-[3-(trifluoromethyl)benzyl]pyrazolo[1,5-a]pyrimidine-3-carboxylic acid (Intermediate 10) and 3-aminopropan-1-ol, 9.1 mg (56%) of the title compound was obtained. MS (ESI+) calcd for C₂₀H₂₁F₃N₄O₂ 406.1617, found 406.1618.

### Example 36

### Synthesis of 6-(3,5-dichlorobenzyl)-N-(2-methoxyethyl)-5,7-dimethylpyrazolo[1,5-a]pyrimidine-3-carboxamide

Following the procedure described in Example 26, but using 6-(3,5-dichlorobenzyl)-5,7-dimethylpyrazolo[1,5-a]pyrimidine-3-carboxylic acid (Intermediate 11) and 2-methoxyethanamine, 8.9 mg (55%) of the title compound was obtained. MS (ESI+) calcd for C₁₉H₂₀Cl₂N₄O₂ 406.0963, found 406.096.

### Example 37

### Synthesis of N-(2-methoxyethyl)-5,7-dimethyl-6-[3-(trifluoromethoxy)benzyl]-pyrazolo[1,5-a]pyrimidine-3-carboxamide

Following the procedure described in Example 26, but using 5,7-dimethyl-6-[3-(trifluoromethoxy)benzyl]pyrazolo[1,5-a]pyrimidine-3-carboxylic acid (Intermediate 12) and 2-methoxyethanamine, 3.0 mg (18%) of the title compound was obtained. MS (ESI+) calcd for C₂₀H₂₁F₃N₄O₃ 422.1566, found 422.1571.

### Example 38

### Synthesis of 6-(2,5-dichlorobenzyl)-N-(2-methoxyethyl)-5,7-dimethylpyrazolo[1,5-a]pyrimidine-3-carboxamide

Following the procedure described in Example 26, but using 6-(2,5-dichlorobenzyl)-5,7-dimethylpyrazolo[1,5-a]pyrimidine-3-carboxylic acid (Intermediate 13) and 2-methoxyethanamine, 6.2 mg (38%) of the title compound was obtained. MS (ESI+) calcd for C₁₉H₂₀Cl₂N₄O₂ 406.0963, found 406.0962.

### Example 39

### Synthesis of 6-(4-bromobenzyl)-N-(2-methoxyethyl)-5,7-dimethylpyrazolo[1,5-a]-pyrimidine-3-carboxamide

Following the procedure described in Example 26, but using 6-(4-bromobenzyl)-5,7-dimethylpyrazolo[1,5-a]pyrimidine-3-carboxylic acid (Intermediate 14) and 2-methoxyethanamine, 6.1 mg (37%) of the title compound was obtained. MS (ESI+) calcd for C₁₉H₂₁BrN₄O₂ 416.0848, found 416.0848.

### Example 40

### Synthesis of N-[2-(acetylamino)ethyl]-6-[3-(benzyloxy)benzyl]-5,7-dimethyl-pyrazolo[1,5-a]pyrimidine-3-carboxamide

Following the procedure described in Example 26, but using 6-[3-(benzyloxy)benzyl]-5,7-dimethylpyrazolo[1,5-a]pyrimidine-3-carboxylic acid (Intermediate 15) and N-(2-aminoethyl)acetamide, 4.4 mg (23%) of the title compound was obtained. MS (ESI+) calcd for C₂₇H₂₉N₅O₃ 471.227, found 471.227.

### Example 41

### Synthesis of N-(2-methoxyethyl)-5,7-dimethyl-6-(2-naphthylmethyl)pyrazolo[1,5-a]pyrimidine-3-carboxamide

2-Bromomethylnaphthalene (553 mg, 2.5 mmol) was dissolved in DMF (20 mL), followed by pentane-2,4-dione (500 mg, 5.0 mmol) and Li₂CO₃ (370 mg, 0.5 mmol). The reaction was heated at 80° C. overnight, then cooled to rt, filtrated through celite and evaporated to 504 mg (84%) 3-(2-naphthylmethyl)pentane-2,4-dione as a yellowish gum.

3-(2-Naphthylmethyl)pentane-2,4-dione (504 mg, 2.1 mmol) was dissolved in EtOH (50 mL), followed by ethyl 5-amino-3-methyl-1H-pyrazole-4-carboxylate (362 mg, 2.3 mmol) and conc HCl (0.125 mL). The mixture was heated at 90° C. for 2.5 h, 1 M KOH (15 mL) added, and heating at reflux continued for 45 min. The mixture was cooled to rt, then concentrated to a third of volume, which on acidification with 1 M HCl precipitated 250 mg (40%) 5,7-dimethyl-6-(2-naphthylmethyl)pyrazolo[1,5-a]pyrimidine-3-carboxylic acid as a brown solid.

5,7-Dimethyl-6-(3-methylbenzyl)pyrazolo[1,5-a]pyrimidine-3-carboxylic acid (25 mg, 0.085 mmol) was dissolved in DMF (5 mL), followed by TBTU (35 mg, 1.1 mmol), triethylamine (11 mg, 1.1 mmol) and 2-methoxyethylamine (7.0 mg, 0.094 mmol). The mixture was stirred at rt for 4 h and then evaporated. The crude product was purified using reversed phase preparative HPLC giving 1.4 mg (4%) N-(2-methoxyethyl)-5,7-dimethyl-6-(2-naphthylmethyl)pyrazolo[1,5-a]pyrimidine-3-carboxamide as a yellow gum. MS (ESI+) calcd for C₂₃H₂₄N₄O₂ 388.1899, found 388.1901.

### Example 42

### Synthesis of 5,7-dimethyl-6-(3-methylbenzyl)-N-{2-[(pyrazin-2-ylcarbonyl)amino]-ethyl}pyrazolo[1,5-a]pyrimidine-3-carboxamide

3-Methylbenzylbromide (463 mg, 2.5 mmol) was dissolved in DMF (20 mL), followed by pentane-2,4-dione (500 mg, 5.0 mmol) and Li₂CO₃ (370 mg, 0.5 mmol). The reaction was heated at 80° C. overnight, cooled to rt, filtrated through celite and evaporated to give 435 mg (85%) 3-(3-methylbenzyl)pentane-2,4-dione as a yellowish gum.

3-(3-Methylbenzyl)pentane-2,4-dione (435 mg, 2.1 mmol) was dissolved in EtOH (50 mL), followed by ethyl 5-amino-3-methyl-1H-pyrazole-4-carboxylate (362 mg, 2.3 mmol) and conc HCl (0.125 mL). The mixture was heated at 90° C. for 2.5 h, 1 M KOH (15 mL) added and heating at reflux continued for 45 min. The mixture was cooled to rt, then concentrated to a third of volume, which on acidification with 1 M HCl precipitated 250 mg (40%) 5,7-dimethyl-6-(3-methylbenzyl)pyrazolo[1,5-a]pyrimidine-3-carboxylic acid as a brown solid.

5,7-dimethyl-6-(3-methylbenzyl)pyrazolo[1,5-a]pyrimidine-3-carboxylic acid (150 mg, 0.505 mmol) was dissolved in DMF (15 mL), followed by N-Boc-ethylenediamine (97 mg, 0.606 mmol), TBTU (244 mg, 0.76 mmol) and triethylamine (76 mg, 0.76 mmol). The mixture was stirred at rt for 4 h then evaporated. The crude product was purified using reversed phase preparative HPLC and then deprotected by addition of DCM/TFA (1:1, 10 mL) and stirring at rt for 30 min, giving 165 mg (96%) N-[2-aminoethyl]-5,7-dimethyl-6-(3-methylbenzyl)pyrazolo[1,5-a]pyrimidine-3-carboxamide as a yellow gum.

N-[2-aminoethyl]-5,7-dimethyl-6-(3-methylbenzyl)pyrazolo[1,5-a]pyrimidine-3-carboxamide (20 mg, 0.059 mmol) from the previous step was dissolved in DMF (3 mL), followed by 2-pyrazinecarboxylic acid (11 mg, 0.089 mmol), TBTU (31 mg, 0.096 mmol) and TEA (9.6 mg, 0.096 mmol). The mixture was stirred at rt overnight, then purified using reversed phase preparative HPLC to 8.0 mg (30%) 5,7-dimethyl-6-(3-methylbenzyl)-N-{2-[(pyrazin-2-ylcarbonyl)amino]ethyl}pyrazolo[1,5-a]pyrimidine-3-carboxamide as an off-white solid. MS (ESI+) calcd for C₂₄H₂₅N₇O₂ 443.207, found 443.2068.

### Example 43

### Synthesis of N-[2-(benzyloxy)ethyl]-5,7-dimethyl-6-[3-(trifluoromethoxy)benzyl]-pyrazolo[1,5-a]pyrimidine-3-carboxamide

Following the procedure described in Example 26, but using 5,7-Dimethyl-6-[3-(trifluoromethoxy)benzyl]pyrazolo[1,5-a]pyrimidine-3-carboxylic acid (Intermediate 12) and [2-(benzyloxy)ethyl]amine hydrochloride, 1.0 mg (5.0%) of the title compound was obtained as a white solid. MS (ESI+) calcd for C₂₆H₂₅F₃N₄O₃ 498.1879, found 498.188.

### Example 44

### Synthesis of N-(3-amino-3-oxopropyl)-6-(3,5-dichlorobenzyl)-5,7-dimethyl-pyrazolo[1,5-a]pyrimidine-3-carboxamide

6-(3,5-Dichlorobenzyl)-5,7-dimethylpyrazolo[1,5-a]pyrimidine-3-carboxylic acid (Intermediate 11) (10 mg, 0.030 mmol) was dissolved in DMF (2 mL), followed by beta-alaninamide hydrochloride (4.5 mg, 0.036 mmol), TBTU (12 mg, 0.040 mmol) and triethylamine (4.0 mg, 0.040 mmol). The mixture was stirred at rt overnight, evaporated and purified using reversed phase preparative HPLC to 5.4 mg (43%) N-(3-amino-3-oxopropyl)-6-(3,5-dichlorobenzyl)-5,7-dimethylpyrazolo[1,5-a]pyrimidine-3-carboxamide as a white solid. MS (ESI+) calcd for C₁₉H₁₉Cl₂N₅O₂ 419.0916, found 419.0913.

### Example 45

### Synthesis of 5,7-dimethyl-N-{2-[(pyridin-3-ylcarbonyl)amino]ethyl}-6-[3-(trifluoro-methoxy)benzyl]pyrazolo[1,5-a]pyrimidine-3-carboxamide

Following the procedure described in Example 44, but using 5,7-dimethyl-6-[3-(trifluoromethoxy)benzyl]pyrazolo[1,5-a]pyrimidine-3-carboxylic acid (Intermediate 12) and N-(2-amino-ethyl)-nicotinamide dihydrochloride (8.5 mg, 0.036 mmol), 6.9 mg (45%) of the title compound was obtained as a light yellow solid. MS (ESI+) calcd for C₂₅H₂₃F₃N₆O₃ 512.1784, found 512.1778.

### Example 46

### Synthesis of 6-[4-fluoro-3-(trifluoromethoxy)benzyl]-N-[2-(2-hydroxyethoxy)ethyl]-5,7-dimethylpyrazolo[1,5-a]pyrimidine-3-carboxamide

5-(Bromomethyl)-2-fluorophenyl trifluoromethyl ether (500 mg, 1.8 mmol) was mixed with pentane-2,4-dione (360 mg, 3.6 mmol) and Li₂CO₃ (27 mg, 0.36 mmol) and DMF (20 mL). The reaction mixture was heated at 80° C. for 2 h then cooled to rt, filtrated through celite and evaporated to 520 mg (98%) 3-[4-fluoro-3-(trifluoromethoxy)-benzyl]pentane-2,4-dione as a light yellow oil.

3-[4-Fluoro-3-(trifluoromethoxy)benzyl]pentane-2,4-dione (520 mg, 1.7 mmol) was dissolved in EtOH (15 mL), followed by ethyl 5-amino-3-methyl-1H-pyrazole-4-carboxylate (362 mg, 2.3 mmol) and conc HCl (0.125 mL). The mixture was heated at 90° C. for 2.5 h and then cooled to rt. 1 M KOH (15 mL) was added and heating at reflux continued for 45 min. The reaction mixture was concentrated to a third of volume and then the acid was precipitated using 2 M HCl, giving 450 mg (69%) 6-[4-fluoro-3-(trifluoromethoxyl)benzyl]-5,7-dimethylpyrazolo[1,5-a]pyrimidine-3-carboxylic acid as a yellow solid.

Following the procedure described in Example 44, but using 6-[4-fluoro-3-(trifluoromethoxyl)benzyl]-5,7-dimethylpyrazolo[1,5-a]pyrimidine-3-carboxylic acid from the previous step and 2-(2-aminoethoxy)ethanol, 0.5 mg (2%) of the title compound was obtained as a white solid. MS (ESI+) calcd for C₂₁H₂₂F₄N₄O₄ 470.1577, found 470.1587.

### Example 47

### Synthesis of 6-[4-fluoro-3-(trifluoromethyl)benzyl]-N-[2-(2-hydroxyethoxy)ethyl]-5,7-dimethylpyrazolo[1,5-a]pyrimidine-3-carboxamide

4-(Bromomethyl)-1-fluoro-2-(trifluoromethyl)benzene (771 mg, 3.0 mmol) was mixed with pentane-2,4-dione (600 mg, 6.0 mmol) and Li₂CO₃ (22 mg, 0.3 mmol) and DMF (15 mL). The reaction mixture was heated at 80° C. for 2 h then cooled to rt, filtrated through celite and evaporated to 800 mg (96%) 3-[4-fluoro-3-(trifluoromethyl)-benzyl]pentane-2,4-dione as a brown gum. 3-[4-Fluoro-3-(trifluoromethyl)benzyl]pentane-2,4-dione (800 mg, 2.9 mmol) was dissolved in EtOH (15 mL), followed by ethyl 5-amino-3-methyl-1H-pyrazole-4-carboxylate (465 mg, 3.0 mmol) and conc HCl (0.25 mL). The mixture was heated at 90° C. for 2.5 h and then cooled to rt. 1 M KOH (15 mL) was added and heating at reflux continued for 45 min. The reaction mixture was concentrated to a third of volume and then the acid was precipitated using 2 M HCl, giving 750 mg (70%) 6-[4-fluoro-3-(trifluoromethyl)benzyl]-5,7-dimethylpyrazolo[1,5-a]pyrimidine-3-carboxylic acid as a brown solid.

Following the procedure described in Example 44, but using 6-[4-fluoro-3-(trifluoromethyl)benzyl]-5,7-dimethylpyrazolo[1,5-a]pyrimidine-3-carboxylic acid from the previous step and 2-(2-aminoethoxy)ethanol, 0.7 mg (2%) of the title compound was obtained as a yellow gum. MS (ESI+) calcd for C₂₁H₂₂F₄N₄O₃ 454.1628, found 454.1626.

### BIOLOGICAL EXAMPLES

### Background to Assay Methodology

Several assay methods for measuring stearoyl-CoA desaturase activity have been described in the literature. Thin layer chromatography, gas chromatography or HPLC methods are commonly used for separation of substrates and products, e.g. stearoyl-CoA and oleyl-CoA, following the enzymatic reaction [see e.g. Henderson & Henderson (1992) In Lipid analysis: A practical approach. Oxford University Press, New York and Tokyo, editor S. Hamilton, pages 65-111]. However, these assays are time-consuming and not amenable to higher throughputs. Spectrophotometric assays in which the SCD activity is followed indirectly by measuring the reoxidation of reduced cytochrome B5 could be applied [Strittmatter (1978) Purification of cytochrome B5. Meth. Enzymol. 52, 97-101] although the fast reoxidation rate complicates the automation of such assays. It may be possible to achieve a reasonable throughput given auto-injectors and fast readers or alternative systems that allow parallel processing of multiple samples, but spectroscopic assays based on near-UV wavelength measurements also have the added disadvantage of being prone to artifacts by colored and autofluorescent compounds.

Another measure of SCD activity was introduced by Talamo and Bloch in 1969 [Talamo & Bloch (1969) Anal. Biochem. 29, 300-304]. This method is based on the quantification of a second product of the desaturase reaction, i.e. the water molecule that is released in the desaturase reaction. The quantification is based on the use of a long chain acyl-CoA substrate, e.g. stearoyl-CoA, that is specifically labeled with tritium in positions 9 and 10 of the carbon chain such that the released water is also tritiated ([³H]-H₂O). The remaining [³H]-stearoyl-CoA as well as the product [³H]-oleyl-CoA must then be separated from the solution before the tritiated water content can be measured by means of liquid scintillation. Talamo and Bloch acid precipitated the long chain acyl-CoAs followed by filtration to achieve this separation, but this separation can also be achieved by means of centrifugation instead of filtration [Johnson & Guhr (1971) Lipids 6, 78-84]. An alternative procedure that involves precipitation by ethanol and activated charcoal followed by centrifugation has also been described [Shanklin and Somerville (1991) Proc. Natl. Acad. Sci. USA 88, 2510-2514]. Based on these studies it is clear that near perfect separation is required for optimal assay performance. When applying this assay it is important to recognize that the apparent desaturation rate is impacted by isotope effects as described by Johnson and Gurr in 1971 [Johnson & Guhr (1971) Lipids 6, 78-84]. Thus whereas the assay serves as an excellent measure of relative SCD activity it must be calibrated using other methods when absolute measures of enzyme activity are needed. The pros and cons of this assay have also been summarized in the literature [Gurr & Robinson (1972) Anal. Biochem. 47, 146-156]. An abundant source of stearoyl-CoA desaturase activity can be found in microsomal preparations from the liver of rats that have been subjected to a fasting-refeeding procedure on a low fat/high carbohydrate diet [reviewed in Ntambi (1999) J. Lipid Res. 40, 1549-1558]. However, microsomal preparations are not a pure source of SCD activity and this means that the added stearoyl-CoA substrate is subject also to other enzymatic processes. It is therefore essential to include reagents that allow regeneration of the stearoyl-CoA substrate as described by Bertram and Erwin [Bertram & Erwin (1981) J. Protozool. 8, 127-131].

The tritium release assay for the measurement of SCD activity is thus well documented in the literature. Descriptions on how these finding have been used to produce standard screening assays in 96-well plates are also available [Brownlie, Hayden, Attie, Ntambi, Gray-Keller, & Miyazaki (2001) WO 01/62954; Wu, Gallipoli, Gallagher, & Gardell (2004) WO 2004/04776]. We have adopted the tritium release assay to a 384-well format to improve throughput even further. The assay is based on the findings made decades ago and hence is available to anyone skilled in the art of assay automation and high throughput screening.

Description of Screening Assay for the Identification and Characterization of Test Compounds that Inhibit Stearoyl-CoA Desaturase Activity

Microsomal preparations were prepared from the livers of Male Sprague-Dawley rats that had been fasted and then refed a low fat/high carbohydrate diet. The preparation of microsomes was adopted from Seifried and Gaylor [Seifried & Gaylor (1976) J. Biol. Chem. 251, 7468-7473]. Confirmation of compound activity on human material was made based on microsomal preparations from HepG2 cells. All other reagents were purchased from commercial sources. The assay was run in 96 or 384-well microtiter plates by consecutive additions of a test compound solution, a microsomal preparation solution and a substrate containing solution. The final concentrations of all reagents in a total assay volume of 40 μl per well (in the 384-well plate format) were:
0.11 μM [³H]-stearoyl-CoA
50 nM stearoyl-CoA
0.032 mg/ml rat liver microsomes (total protein content)
2 mM NADH
220 mM sucrose
44 mM NaH₂PO₄ pH adjusted to 6.8
130 mM KCl
1.3 mM GSH
0.05 mM CoA
0.1% BSA
0.29 mM nicotine amide
15 mM NaF
1.1 mM ATP
4.9 mM MgCl2
0.002% Tween-20
A test compound at various concentrations (which also adds 0.5-2% DMSO to the final solution)

The test compounds were pre-incubated for 20 minutes with the microsomal preparation prior to starting the reaction by the addition of substrate. The enzymatic reaction was allowed to proceed for 20 minutes and then optionally slowed by an addition of 40 μl of a 2% DMSO solution in water containing a known inhibitor of SCD activity. The solutions were mixed and then 70 μl of the total 80 μl were transferred to a filter plate containing predispensed activated charcoal. The plate was then centrifuged and the filtrate collected in a collector plate to which 40 μl of Optiphase Supermix was added per well. Following an 18 h equilibration time at room temperature the plate was read in a Trilux MicroBeta (two minutes counting time per well). On all assay occasions controls were included on each plate to define the values for uninhibited and fully inhibited reactions and these values were used to calculate the % inhibition of the enzymatic reaction at any given compound concentration. The inhibitory potency or IC₅₀ values of test compounds on SCD activity were defined by applying the same assay in the presence of sub-nM to sub-mM compound concentrations. Examples included herein have IC₅₀ values in the range of 1 nM to 5 μM (see Table I for exemplary data) as measured using the above described assay or in the equivalent assay in a 96-well microtiter plate format.

TABLE I
IC₅₀ values for SCD inhibition

| Compound | IC₅₀ (μM) |
|---|---|
| 6-benzyl-N-(2-methoxyethyl)-5,7- | 1.0 ± 0.40 |
| dimethylpyrazolo[1,5-a]-pyrimidine-3-carboxamide | |
| 6-(3-bromobenzyl)-5,7-dimethyl-N-(tetrahydrofuran-2- | 0.14 ± 0.067 |
| ylmethyl)pyrazolo[1,5-a]pyrimidine-3-carboxamide | |
| N-(3-ethoxypropyl)-6-(3-fluorobenzyl)-5,7- | 0.70 ± 0.11 |
| dimethylpyrazolo[1,5-a]pyrimidine-3-carboxamide | |
| N-(2-acetamidoethyl)-6-(3-methoxybenzyl)-5,7- | 0.83 ± 0.24 |
| dimethylpyrazolo[1,5-a]pyrimidine-3-carboxamide | |

1. A pharmaceutical composition comprising a compound of the Formula I 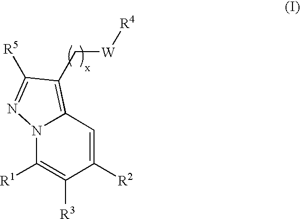
including pharmaceutically acceptable salts, solvates, hydrates, geometrical isomers, tautomers, optical isomers, and N-oxides thereof, wherein:
x is 0 or 1;
W is a direct bond, —C(O)N(R⁶)—, —N(R⁶)C(O)—, —C(O)O—, —OC(O)—, —O—, —N(R⁶)C(O)N(R⁶)—, —N(R⁶)—;
wherein each R⁶ is independently hydrogen, C₁-C₃ alkyl, or C₃-C₈ alkoxyalkyl;
One of R¹, R² and R³ is Y-R¹⁸, and the other two are independently selected from the group consisting of hydrogen, C₁-C₃ alkyl, C₁-C₃ fluoroalkyl;
Y is selected from the group consisting of —S—, —O—, and C₁-C₃ alkylene, wherein C₁-C₃ alkylene is optionally monosubstituted with hydroxy or oxo, or is partly or fully fluorinated;
R¹⁸ is aryl or heteroaryl, which is optionally substituted in one or more positions;
R⁴ is selected from the group consisting of hydrogen, C₁-C₆ alkyl, C₂-C₆ alkenyl, C₃-C₆ alkynyl, C₁-C₆ fluoroalkyl, C₃-C₈ alkylthioalkyl, C₃-C₆ cyanoalkyl, C₈-C₁₂ arylalkyl, C₃-C₆ cycloalkyl, C₃-C₈ heteroaryl, aryl, C₅-C₁₀ heteroarylalkyl, C₄-C₆ heterocyclylalkyl, and C₃-C₈ heterocyclyl, provided that said heterocyclyl is bonded via a ring carbon;
or R⁴ is C₁₋₆ alkylene-V—R⁷;
wherein V is selected from the group consisting of —N(R¹⁵)—, —C(O)N(R¹⁵)—, —C(O)O—, —OC(O)—, —C(O)—, —O—, —N(R¹⁵)C(O)—, —N(R¹⁵)C(O)N(R¹⁵)—, —S—, —S(O)—, —S(O)₂—, —S(O)₂N(R¹⁵)—, and —N(R¹⁵)S(O)₂—;
and wherein each R⁷ and each R¹⁵ are independently selected from the group consisting of hydrogen, C₁-C₅ alkyl, hydroxy-C₁-C₅ alkyl, aryl-C₁-C₅ alkyl, heteroaryl-C₁-C₅ alkyl, heteroaryl, heterocyclyl, C₄-C₈ cycloalkylalkyl, C₃-C₈ cycloalkyl and C₁-C₅ fluoroalkyl, provided that when V is selected from —S—, —S(O)— or —S(O)₂—, R⁷ is not hydrogen;
or R⁴ and an R⁶ together form a C₃-C₅ heterocyclyl ring; and
R⁵ is hydrogen or C₁-C₃ alkyl;
provided that the said compound is not selected from the group consisting of:
N-cyclopentyl-5,7-dimethyl-6-(2,4,6-trimethylbenzyl)-pyrazolo[1,5-a]-pyrimidine-3-carboxamide; and
6-(4-chlorobenzyl-5,7-dimethyl-N-(1-methylethyl)-pyrazolo[1,5-a]-pyrimidine-3-carboxamide;
and a pharmaceutically acceptable diluent or carrier.

2. The composition according to claim 1, wherein x is 0 and W is —C(O)NH—.

3. The composition according to claim 1, wherein R¹ is methyl.

4. The composition according to claim 1, wherein R² is methyl.

5. The composition according to claim 1, wherein R³ is C₇-C₁₂ arylalkyl.

6. The composition according to claim 1, wherein R⁴ is C₃-C₈ alkoxyalkyl, C₂-C₆ hydroxyalkyl, C₃-C₈ alkylthioalkyl, or C₄-C₆ heterocyclylalkyl.

7. The composition according to claim 1, wherein R⁴ is
C₁-C₆ alkylene-V—R⁷;
wherein V is selected from the group consisting of —N(R¹⁵)C(O)—, —C(O)N(R¹⁵)—, —O—, and —S(O)—,
and wherein each R⁷ and each R¹⁵ are independently selected from the group consisting of hydrogen, C₁-C₅ alkyl, hydroxy-C₁-C₅ alkyl, C₂-C₅ fluoroalkyl, C₃-C₆ cycloalkyl and heteroaryl.

8. The composition according to claim 1, wherein R⁴ and R⁶ together form a C₃-C₅ heterocyclyl ring.

9. The composition according to claim 1, wherein R⁵ is H.

10. A pharmaceutical composition comprising a compound of the Formula I 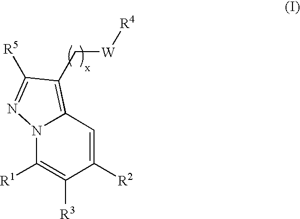
including pharmaceutically acceptable salts, solvates, hydrates, geometrical isomers, tautomers, optical isomers, and N-oxides thereof, wherein:
x is 0 or 1;
W is a direct bond, —C(O)N(R⁶)—, —N(R⁶)C(O)—, —C(O)O—, —OC(O)—, —N(R⁶)C(O)N(R⁶)—, —N(R⁶)—;
wherein each R⁶ is independently hydrogen, C₁-C₃ alkyl, or C₃-C₈ alkoxyalkyl;
R¹ and R² are each independently selected from the group consisting of hydrogen, C₁-C₃ alkyl, and C₁-C₃ fluoroalkyl;
R³ is C₇-C₁₂ arylalkyl or C₃-C₁₀heteroarylalkyl;
R⁴ is selected from the group consisting of hydrogen, C₁-C₆ alkyl, C₂-C₆ alkenyl, C₃-C₆ alkynyl, C₁-C₆ fluoroalkyl, C₃-C₈ alkoxyalkyl, C₁-C₆ hydroxyalkyl, C₃-C₈ alkylthioalkyl, C₃-C₆ cyanoalkyl, C₈-C₁₂ arylalkyl, C₃-C₆ cycloalkyl, C₃-C₈ heteroaryl, aryl, C₄-C₆ heterocyclylalkyl, and C₃-C₉ heterocyclyl, provided that said heterocyclyl is bonded via a ring carbon;
or R⁴ is C₁₋₆ alkylene-V—R⁷;
wherein V is selected from the group consisting of —N(R¹⁵)—, —C(O)N(R¹⁵)—, —C(O)O—, and —OC(O)—, —C(O)—, —N(R¹⁵)C(O)—, —N(R¹⁵)C(O)N(R¹⁵)—, —S(O)—, —S(O)₂—, —S(O)₂N(R¹⁵)—, —N(R¹⁵)S(O)₂—;
and wherein each R⁷ and each R¹⁵ are independently selected from the group consisting of hydrogen, C₁-C₅ alkyl, C₄-C₈ cycloalkylalkyl, C₃-C₈ cycloalkyl and C₁-C₅ fluoroalkyl, provided that when V is selected from —S(O)— or —S(O)₂—, R⁷ is not hydrogen;
or R⁴ and an R⁶ together form a C₃-C₅ heterocyclyl ring; and
R⁵ is hydrogen or C₁-C₃ alkyl;
provided that the said compound is not selected from the group consisting of:
N-cyclopentyl-5,7-dimethyl-6-(2,4,6-trimethylbenzyl)-pyrazolo[1,5-a]-pyrimidine-3-carboxamide; and
6-(4-chlorobenzyl-5,7-dimethyl-N-(1-methylethyl)-pyrazolo[1,5-a]-pyrimidine-3-carboxamide;
and a pharmaceutically acceptable diluent or carrier.

11. The composition according to claim 1 wherein the compound is of the Formula II 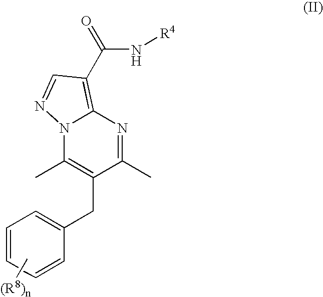
wherein:
R⁴ is as defined in claim 1;
n is 0, 1, 2 or 3; and
each R⁸ is independently selected from the group consisting of fluoro, chloro, bromo, methyl, ethyl, methoxy, ethoxy, hydroxy, hydroxymethyl, trifluoromethyl, trifluoromethoxy, methylthio, trifluoromethylthio and benzyloxy; or two substituents R⁹ together form a saturated or unsaturated, aliphatic or heterocyclic ring;
provided that the said compound is not selected from the group consisting of:
N-cyclopentyl-5,7-dimethyl-6-(2,4,6-trimethylbenzyl)-pyrazolo[1,5-a]-pyrimidine-3-carboxamide; and
6-(4-chlorobenzyl-5,7-dimethyl-N-(1-methylethyl)-pyrazolo[1,5-a]-pyrimidine-3-carboxamide.

12. The composition according to claim 11, wherein R⁴ is C₃-C₈alkoxyalkyl, C₂-C₆ hydroxyalkyl, C₃-C₈ alkylthioalkyl, or C₄-C₆ heterocyclylalkyl.

13. The composition according to claim 12, wherein R⁴ is 2-methoxyethyl, 3-ethoxypropyl, 3-isopropoxypropyl, tetrahydrofuran-2-ylmethyl, or 2-(1,3-dioxolan-2-yl)ethyl.

14. The composition according to claim 11, wherein R⁴ is
C₁-C₆ alkylene-V—R⁷;
wherein V is selected from the group consisting of —N(R¹⁵)C(O)—, C(O)N(R¹⁵)—, —O—, and —S(O)—,
and wherein each R⁷ and each R¹⁵ are independently selected from the group consisting of hydrogen, C₁-C₅ alkyl, hydroxy-C₁-C₅ alkyl, C₂-C₅ fluoroalkyl, C₃-C₆ cycloalkyl and heteroaryl.

15. The composition according to claim 14, wherein
R⁴ is —(CH₂)ₚ—NHC(O)R⁹
wherein R⁹ is C₁-C₃ alkyl; and p is 2, 3, or 4;
or R⁴ is —(CH₂)_{z}—C(O)NR¹⁷R¹⁷;
wherein each R¹⁷ is independently hydrogen or C₁-C₃ alkyl; and z is 1 or 2.

16. The composition according to claim 1, wherein the compound is selected from the group consisting of:
6-Benzyl-N-(2-methoxyethyl)-5,7-dimethylpyrazolo[1,5-a]pyrimidine-3-carboxamide;
6-(3-Bromobenzyl)-N-(2-methoxyethyl)-5,7-dimethylpyrazolo[1,5-a]-pyrimidine-3-carboxamide;
6-(2-Fluorobenzyl)-N-(2-methoxyethyl)-5,7-dimethylpyrazolo[1,5-a]-pyrimidine-3-carboxamide;
6-(2-Fluorobenzyl)-N-(3-methoxypropyl)-5,7-dimethylpyrazolo[1,5-a]-pyrimidine-3-carboxamide;
6-(2-Bromobenzyl)-N-(3-methoxypropyl)-5,7-dimethylpyrazolo[1,5-a]-pyrimidine-3-carboxamide;
6-(3-Bromobenzyl)-5,7-dimethyl-N-(tetrahydrofuran-2-ylmethyl)-pyrazolo[1,5-a]pyrimidine-3-carboxamide;
6-(Mesitylmethyl)-N-(2-methoxyethyl)-5,7-dimethylpyrazolo[1,5-a]-pyrimidine-3-carboxamide;
N-(2-Methoxyethyl)-5,7-dimethyl-6-(2-methylbenzyl)pyrazolo[1,5-a]-pyrimidine-3-carboxamide;
6-(2,5-Dimethylbenzyl)-N-(2-methoxyethyl)-5,7-dimethylpyrazolo[1,5-a]-pyrimidine-3-carboxamide;
6-(4-Chlorobenzyl)-N-(2-methoxyethyl)-5,7-dimethylpyrazolo[1,5-a]-pyrimidine-3-carboxamide;
6-(2-Chlorobenzyl)-N-(2-methoxyethyl)-5,7-dimethylpyrazolo[1,5-a]-pyrimidine-3-carboxamide;
6-(3-Chlorobenzyl)-N-(2-methoxyethyl)-5,7-dimethylpyrazolo[1,5-a]-pyrimidine-3-carboxamide;
5,7-Dimethyl-6-(2-methylbenzyl)-N-(tetrahydrofuran-2-ylmethyl)-pyrazolo[1,5-a]pyrimidine-3-carboxamide;
6-(2,5-Dimethylbenzyl)-5,7-dimethyl-N-(tetrahydrofuran-2-ylmethyl)-pyrazolo[1,5-a]pyrimidine-3-carboxamide;
Methyl 6-(3-bromobenzyl)-5,7-dimethylpyrazolo[1,5-a]pyrimidine-3-carboxylate;
6-(4-Fluorobenzyl)-5,7-dimethyl-N-(tetrahydrofuran-2-ylmethyl)pyrazolo[1,5-a]pyrimidine-3-carboxamide;
6-(4-Chlorobenzyl)-N-(3-methoxypropyl)-5,7-dimethylpyrazolo[1,5-a]-pyrimidine-3-carboxamide;
6-(4-Chlorobenzyl)-N-(3-ethoxypropyl)-5,7-dimethylpyrazolo[1,5-a]-pyrimidine-3-carboxamide;
6-(2-Chlorobenzyl)-N-(3-methoxypropyl)-5,7-dimethylpyrazolo[1,5-a]-pyrimidine-3-carboxamide;
6-(3-Chlorobenzyl)-N-(3-methoxypropyl)-5,7-dimethylpyrazolo[1,5-a]-pyrimidine-3-carboxamide;
6-(3-Chlorobenzyl)-N-(3-ethoxypropyl)-5,7-dimethylpyrazolo[1,5-a]-pyrimidine-3-carboxamide;
6-(2-Chlorobenzyl)-5,7-dimethyl-N-(tetrahydrofuran-2-ylmethyl)-pyrazolo[1,5-a]pyrimidine-3-carboxamide;
6-(2-Chloro-4-fluorobenzyl)-5,7-dimethyl-N-(tetrahydrofuran-2-ylmethyl)-pyrazolo[1,5-a]pyrimidine-3-carboxamide;
6-(2-Chloro-6-fluorobenzyl)-5,7-dimethyl-N-(tetrahydrofuran-2-ylmethyl)-pyrazolo[1,5-a]pyrimidine-3-carboxamide;
N-(3-Ethoxypropyl)-5,7-dimethyl-6-(2-methylbenzyl)pyrazolo[1,5-a]-pyrimidine-3-carboxamide;
N-(3-Methoxypropyl)-5,7-dimethyl-6-(2-methylbenzyl)pyrazolo[1,5-a]-pyrimidine-3-carboxamide;
N-(3-Ethoxypropyl)-6-(3-methoxybenzyl)-5,7-dimethylpyrazolo[1,5-a]-pyrimidine-3-carboxamide;
6-(3-Methoxybenzyl)-N-(2-methoxyethyl)-5,7-dimethylpyrazolo[1,5-a]-pyrimidine-3-carboxamide;
6-(3-Methoxybenzyl)-N-(3-methoxypropyl)-5,7-dimethylpyrazolo[1,5-a]-pyrimidine-3-carboxamide;
6-(3-Cyanobenzyl)-N-(3-methoxypropyl)-5,7-dimethylpyrazolo[1,5-a]-pyrimidine-3-carboxamide;
N-(3-Ethoxypropyl)-6-(3-fluorobenzyl)-5,7-dimethylpyrazolo[1,5-a]-pyrimidine-3-carboxamide;
6-(3-Fluorobenzyl)-N-(2-methoxyethyl)-5,7-dimethylpyrazolo[1,5-a]-pyrimidine-3-carboxamide;
6-(3-Fluorobenzyl)-N-(3-methoxypropyl)-5,7-dimethylpyrazolo[1,5-a]-pyrimidine-3-carboxamide;
6-Benzyl-N-(3-ethoxypropyl)-5,7-dimethylpyrazolo[1,5-a]pyrimidine-3-carboxamide;
6-Benzyl-N-(3-methoxypropyl)-5,7-dimethylpyrazolo[1,5-a]pyrimidine-3-carboxamide;
N-[2-(Acetylamino)ethyl]-5,7-dimethyl-6-(2-methylbenzyl)pyrazolo[1,5-a]-pyrimidine-3-carboxamide;
N-(3-Isopropoxypropyl)-5,7-dimethyl-6-(2-methylbenzyl)pyrazolo[1,5-a]-pyrimidine-3-carboxamide;
N-(2-Amino-2-oxoethyl)-5,7-dimethyl-6-(2-methylbenzyl)pyrazolo[1,5-a]-pyrimidine-3-carboxamide;
6-(3-Chlorobenzyl)-5,7-dimethyl-N-(tetrahydrofuran-2-ylmethyl)pyrazolo-[1,5-a]pyrimidine-3-carboxamide;
6-(3-Chlorobenzyl)-5,7-dimethyl-N-[2-(methylthio)ethyl]pyrazolo[1,5-a]-pyrimidine-3-carboxamide;
N-[2-(Acetylamino)ethyl]-6-(3-methoxybenzyl)-5,7-dimethylpyrazolo[1,5-a]-pyrimidine-3-carboxamide;
N-(3-Isopropoxypropyl)-6-(3-methoxybenzyl)-5,7-dimethylpyrazolo[1,5-a]-pyrimidine-3-carboxamide;
6-(3-Methoxybenzyl)-5,7-dimethyl-N-(tetrahydrofuran-2-ylmethyl)pyrazolo-[1,5-a]pyrimidine-3-carboxamide;
N-[2-(Acetylamino)ethyl]-6-(3-fluorobenzyl)-5,7-dimethylpyrazolo[1,5-a]-pyrimidine-3-carboxamide;
6-(3-Fluorobenzyl)-N-(3-isopropoxypropyl)-5,7-dimethylpyrazolo[1,5-a]-pyrimidine-3-carboxamide;
N-[2-(1,3-Dioxolan-2-yl)ethyl]-6-(3-fluorobenzyl)-5,7-dimethylpyrazolo[1,5-a]pyrimidine-3-carboxamide;
N-[2-(Acetylamino)ethyl]-6-benzyl-5,7-dimethylpyrazolo[1,5-a]pyrimidine-3-carboxamide;
6-Benzyl-N-(3-isopropoxypropyl)-5,7-dimethylpyrazolo[1,5-a]pyrimidine-3-carboxamide;
6-(3-Bromobenzyl)-N-(3-isopropoxypropyl)-5,7-dimethylpyrazolo[1,5-a]-pyrimidine-3-carboxamide;
N-[2-(Acetylamino)ethyl]-6-(3-bromobenzyl)-5,7-dimethylpyrazolo[1,5-a]-pyrimidine-3-carboxamide;
N-(2-Amino-2-oxoethyl)-6-(3-bromobenzyl)-5,7-dimethylpyrazolo[1,5-a]-pyrimidine-3-carboxamide;
6-(3-Bromobenzyl)-N-(2-hydroxyethyl)-5,7-dimethylpyrazolo[1,5-a]-pyrimidine-3-carboxamide;
6-(3-Bromobenzyl)-N-(2-tert-butoxyethyl)-5,7-dimethylpyrazolo[1,5-a]-pyrimidine-3-carboxamide;
6-(3-Bromobenzyl)-N-(2-isopropoxyethyl)-5,7-dimethylpyrazolo[1,5-a]-pyrimidine-3-carboxamide;
6-Benzyl-5,7-dimethyl-N-(tetrahydrofuran-2-ylmethyl)pyrazolo[1,5-a]-pyrimidine-3-carboxamide;
6-(3,4-dichlorobenzyl)-N-[2-(2-hydroxyethoxy)ethyl]-5,7-dimethylpyrazolo-[1,5-a]pyrimidine-3-carboxamide;
5,7-dimethyl-N-[3-(methylamino)-3-oxopropyl]-6-[3-(trifluoromethoxy)-benzyl]pyrazolo[1,5-a]pyrimidine-3-carboxamide;
N-{3-[(2-hydroxyethyl)amino]-3-oxopropyl}-5,7-dimethyl-6-[3-(trifluoro-methoxy)benzyl]pyrazolo[1,5-a]pyrimidine-3-carboxamide;
6-(3-chlorobenzyl)-N-(2-methoxyethyl)-2,5,7-trimethylpyrazolo[1,5-a]-pyrimidine-3-carboxamide;
6-(3-chloro-4-fluorobenzyl)-N-(3-methoxypropyl)-5,7-dimethylpyrazolo[1,5-a]pyrimidine-3-carboxamide;
N-(3-amino-3-oxopropyl)-6-(3-chloro-4-fluorobenzyl)-5,7-dimethylpyrazolo-[1,5-a]pyrimidine-3-carboxamide;
N-(3-methoxypropyl)-5,7-dimethyl-6-[3-(trifluoromethyl)benzyl]pyrazolo-[1,5-a]pyrimidine-3-carboxamide;
5,7-dimethyl-N-[2-(1-methyl-1H-imidazol-4-yl)ethyl]-6-[3-(trifluoromethyl)-benzyl]pyrazolo[1,5-a]pyrimidine-3-carboxamide;
N-(2-amino-2-oxoethyl)-5,7-dimethyl-6-[3-(trifluoromethyl)benzyl]pyrazolo-[1,5-a]pyrimidine-3-carboxamide;
N-(3-hydroxypropyl)-5,7-dimethyl-6-[3-(trifluoromethyl)benzyl]pyrazolo[1,5-a]pyrimidine-3-carboxamide;
6-(3,5-dichlorobenzyl)-N-(2-methoxyethyl)-5,7-dimethylpyrazolo[1,5-a]-pyrimidine-3-carboxamide;
N-(2-methoxyethyl)-5,7-dimethyl-6-[3-(trifluoromethoxy)benzyl]pyrazolo-[1,5-a]pyrimidine-3-carboxamide;
6-(2,5-dichlorobenzyl)-N-(2-methoxyethyl)-5,7-dimethylpyrazolo[1,5-a]-pyrimidine-3-carboxamide;
6-(4-bromobenzyl)-N-(2-methoxyethyl)-5,7-dimethylpyrazolo[1,5-a]-pyrimidine-3-carboxamide;
N-[2-(acetylamino)ethyl]-6-[3-(benzyloxy)benzyl]-5,7-dimethylpyrazolo[1,5-a]pyrimidine-3-carboxamide;
N-(2-methoxyethyl)-5,7-dimethyl-6-(2-naphthylmethyl)pyrazolo[1,5-a]-pyrimidine-3-carboxamide;
5,7-dimethyl-6-(3-methylbenzyl)-N-{2-[(pyrazin-2-ylcarbonyl)amino]ethyl}-pyrazolo[1,5-a]pyrimidine-3-carboxamide;
N-[2-(benzyloxy)ethyl]-5,7-dimethyl-6-[3-(trifluoromethoxy)benzyl]pyrazolo-[1,5-a]pyrimidine-3-carboxamide;
N-(3-amino-3-oxopropyl)-6-(3,5-dichlorobenzyl)-5,7-dimethylpyrazolo[1,5-a]pyrimidine-3-carboxamide;
5,7-dimethyl-N-{2-[(pyridin-3-ylcarbonyl)amino]ethyl}-6-[3-(trifluoro-methoxy)benzyl]pyrazolo[1,5-a]pyrimidine-3-carboxamide;
6-[4-fluoro-3-(trifluoromethoxy)benzyl]-N-[2-(2-hydroxyethoxy)ethyl]-5,7-dimethylpyrazolo[1,5-a]pyrimidine-3-carboxamide; and
6-[4-fluoro-3-(trifluoromethyl)benzyl]-N-[2-(2-hydroxyethoxy)ethyl]-5,7-dimethylpyrazolo[1,5-a]pyrimidine-3-carboxamide.

17. A method for treatment or prevention of cardiovascular diseases, obesity, non-insulin-dependent diabetes mellitus, hypertension, neurological diseases, immune disorders, cancer, essential fatty acid deficiency, acne, psoriasis, rosacea or other skin conditions caused by lipid imbalance, or for treatment of excessive hair growth, e.g. hirsutism, which comprises administering to a mammal, including man, in need of such treatment an effective amount of a composition according to any one of claims 1, 10 or 16.

18. A method for the modulation of stearoyl-CoA desaturase activity which comprises administering to a mammal, including man, in need of such treatment an effective amount of a composition according to any one of claims 1, 10 or 16.

19. A method for the modulation of plasma lipid levels which comprises administering to a mammal, including man, in need of such treatment an effective amount of a composition according to any one of claims 1, 10 or 16.

20. A pharmaceutical formulation according to any one of claims 1, 10 or 16 further comprising an additional active agent.

21. A compound according to Formula III 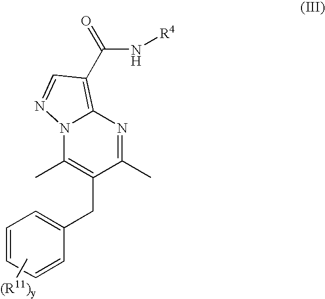
including pharmaceutically acceptable salts, solvates, hydrates, geometrical isomers, tautomers, optical isomers, and N-oxides thereof; wherein:
R¹⁰ is C₁-C₆ alkylene-Z-R¹²;
wherein Z is selected from the group consisting of —N(R⁶)C(O)—, —C(O)N(R¹⁶)—, —N(R¹⁶)C(O)N(R¹⁶)—, —S—, —S(O)—, —S(O)₂—, —S(O)₂N(R¹⁶)—, and —N(R¹⁶)S(O)₂—;
and wherein each R¹² and each R¹⁶ are independently selected from the group consisting of hydrogen, C₁-C₅ alkyl, C₂-C₅ fluoroalkyl, C₃-C₆ cycloalkyl and heteroaryl, provided that when Z is selected from —S—, —S(O)— or —S(O)₂—, R¹² is not hydrogen;
or R¹⁰ is C₁₋₆alkylene-OR¹³;
wherein R¹³ is selected from the group consisting of hydrogen, C₃-C₅ alkyl, hydroxy-C₁-C₅ alkyl, C₂-C₅ fluoroalkyl, C₃-C₆ cycloalkyl and benzyl;
or R¹⁰ is C₂-C₅ fluoroalkyl, C₄-C₆ heterocyclylalkyl or C₃-C₈ heterocyclyl, provided that said heterocyclyl is bonded via a ring carbon atom;
y is 0, 1, 2 or 3; and
R¹¹ is selected from the group consisting of C₁-C₄ alkyl, C₂-C₄ alkenyl, C₃-C₆ cycloalkyl, C₃-C₈ heterocyclyl, aryl, C₁-C₉ heteroaryl, C₁-C₄ fluoroalkyl, C₁-C₄ alkoxy, C₁-C₄ hydroxyalkyl, C₁-C₄ alkylthio, trifluoromethoxy, trifluoromethylthio, benzyloxy, halo, nitro, hydroxy, —OC(O)R⁴, —C(O)R⁴, —C(O)OR¹⁴, —C(O)N(R¹⁴)₂, —N(R¹⁴)₂, —N(R¹⁴)C(O)R¹⁴, —N(R¹⁴)S(O)₂R¹⁴, —S(O)₂N(R⁴)₂, —S(O)R¹⁴ and —S(O)₂R¹⁴;
wherein each R¹⁴ is independently selected from the group consisting of hydrogen, C₁-C₄ alkyl, C₂-C₄ alkenyl, C₂-C₄ fluoroalkyl, C₃-C₆ cycloalkyl, aryl, C₁-C₉ heteroaryl, and C₃-C₈ heterocyclyl, provided that said heterocyclyl is bonded via a ring carbon atom; and provided that when R¹¹ is selected from —S(O)R¹⁴ or —S(O)₂R¹⁴, R¹⁴ is not hydrogen;
or two substituents R¹¹ together form a saturated or unsaturated, aliphatic or heterocyclic ring;
and provided that the said compound is not selected from the group consisting of:
5,7-Dimethyl-6-(4-methylbenzyl)-N-(tetrahydrofuran-2-ylmethyl)pyrazolo-[1,5-a]pyrimidine-3-carboxamide;
5,7-Dimethyl-6-(3-methylbenzyl)-N-(tetrahydrofuran-2-ylmethyl)pyrazolo-[1,5-a]pyrimidine-3-carboxamide;
6-(3-Bromobenzyl)-5,7-dimethyl-N-(tetrahydrofuran-2-ylmethyl)pyrazolo-[1,5-a]pyrimidine-3-carboxamide;
5,7-Dimethyl-6-(2-methylbenzyl)-N-(tetrahydrofuran-2-ylmethyl)pyrazolo-[1,5-a]pyrimidine-3-carboxamide;
6-(2,5-Dimethylbenzyl)-5,7-dimethyl-N-(tetrahydrofuran-2-ylmethyl)-pyrazolo[1,5-a]pyrimidine-3-carboxamide;
6-(4-Fluorobenzyl)-5,7-dimethyl-N-(tetrahydrofuran-2-ylmethyl)pyrazolo-[1,5-a]pyrimidine-3-carboxamide;
6-(2-Chlorobenzyl)-5,7-dimethyl-N-(tetrahydrofuran-2-ylmethyl)pyrazolo-[1,5-a]pyrimidine-3-carboxamide;
6-(2-Chloro-4-fluorobenzyl)-5,7-dimethyl-N-(tetrahydrofuran-2-ylmethyl)-pyrazolo[1,5-a]pyrimidine-3-carboxamide;
6-(2-Chloro-6-fluorobenzyl)-5,7-dimethyl-N-(tetrahydrofuran-2-ylmethyl)-pyrazolo[1,5-a]pyrimidine-3-carboxamide; and
6-(3-Fluorobenzyl)-5,7-dimethyl-N-(tetrahydrofuran-2-ylmethyl)pyrazolo-[1,5-a]pyrimidine-3-carboxamide.

22. A compound according to claim 21 which is selected from the group consisting of:
N-[2-(Acetylamino)ethyl]-5,7-dimethyl-6-(2-methylbenzyl)pyrazolo[1,5-a]-pyrimidine-3-carboxamide;
N-(3-Isopropoxypropyl)-5,7-dimethyl-6-(2-methylbenzyl)pyrazolo[1,5-a]-pyrimidine-3-carboxamide;
N-(2-Amino-2-oxoethyl)-5,7-dimethyl-6-(2-methylbenzyl)pyrazolo[1,5-a]-pyrimidine-3-carboxamide;
6-(3-Chlorobenzyl)-5,7-dimethyl-N-(tetrahydrofuran-2-ylmethyl)pyrazolo-[1,5-a]pyrimidine-3-carboxamide;
6-(3-Chlorobenzyl)-5,7-dimethyl-N-[2-(methylthio)ethyl]pyrazolo[1,5-a]-pyrimidine-3-carboxamide;
N-[2-(Acetylamino)ethyl]-6-(3-methoxybenzyl)-5,7-dimethylpyrazolo[1,5-a]-pyrimidine-3-carboxamide;
N-(3-Isopropoxypropyl)-6-(3-methoxybenzyl)-5,7-dimethylpyrazolo[1,5-a]-pyrimidine-3-carboxamide;
6-(3-Methoxybenzyl)-5,7-dimethyl-N-(tetrahydrofuran-2-ylmethyl)pyrazolo-[1,5-a]pyrimidine-3-carboxamide;
N-[2-(Acetylamino)ethyl]-6-(3-fluorobenzyl)-5,7-dimethylpyrazolo[1,5-a]-pyrimidine-3-carboxamide;
6-(3-Fluorobenzyl)-N-(3-isopropoxypropyl)-5,7-dimethylpyrazolo[1,5-a]-pyrimidine-3-carboxamide;
N-[2-(1,3-Dioxolan-2-yl)ethyl]-6-(3-fluorobenzyl)-5,7-dimethylpyrazolo[1,5-a]pyrimidine-3-carboxamide;
N-[2-(Acetylamino)ethyl]-6-benzyl-5,7-dimethylpyrazolo[1,5-a]pyrimidine-3-carboxamide;
6-Benzyl-N-(3-isopropoxypropyl)-5,7-dimethylpyrazolo[1,5-a]pyrimidine-3-carboxamide;
6-(3-Bromobenzyl)-N-(3-isopropoxypropyl)-5,7-dimethylpyrazolo[1,5-a]-pyrimidine-3-carboxamide;
N-[2-(Acetylamino)ethyl]-6-(3-bromobenzyl)-5,7-dimethylpyrazolo[1,5-a]-pyrimidine-3-carboxamide;
N-(2-Amino-2-oxoethyl)-6-(3-bromobenzyl)-5,7-dimethylpyrazolo[1,5-a]-pyrimidine-3-carboxamide;
6-(3-Bromobenzyl)-N-(2-hydroxyethyl)-5,7-dimethylpyrazolo[1,5-a]-pyrimidine-3-carboxamide;
6-(3-Bromobenzyl)-N-(2-tert-butoxyethyl)-5,7-dimethylpyrazolo[1,5-a]-pyrimidine-3-carboxamide;
6-(3-Bromobenzyl)-N-(2-isopropoxyethyl)-5,7-dimethylpyrazolo[1,5-a]-pyrimidine-3-carboxamide;
6-Benzyl-5,7-dimethyl-N-(tetrahydrofuran-2-ylmethyl)pyrazolo[1,5-a]-pyrimidine-3-carboxamide;
6-(3,4-dichlorobenzyl)-N-[2-(2-hydroxyethoxy)ethyl]-5,7-dimethylpyrazolo-[1,5-a]pyrimidine-3-carboxamide;
5,7-dimethyl-N-[3-(methylamino)-3-oxopropyl]-6-[3-(trifluoromethoxy)-benzyl]pyrazolo[1,5-a]pyrimidine-3-carboxamide;
N-{3-[(2-hydroxyethyl)amino]-3-oxopropyl}-5,7-dimethyl-6-[3-(trifluoro-methoxy)benzyl]pyrazolo[1,5-a]pyrimidine-3-carboxamide;
N-(3-amino-3-oxopropyl)-6-(3-chloro-4-fluorobenzyl)-5,7-dimethylpyrazolo-[1,5-a]pyrimidine-3-carboxamide;
N-(2-amino-2-oxoethyl)-5,7-dimethyl-6-[3-(trifluoromethyl)benzyl]pyrazolo-[1,5-a]pyrimidine-3-carboxamide;
N-(3-hydroxypropyl)-5,7-dimethyl-6-[3-(trifluoromethyl)benzyl]pyrazolo-[1,5-a]pyrimidine-3-carboxamide;
N-[2-(acetylamino)ethyl]-6-[3-(benzyloxy)benzyl]-5,7-dimethylpyrazolo[1,5-a]pyrimidine-3-carboxamide;
5,7-dimethyl-6-(3-methylbenzyl)-N-{2-[(pyrazin-2-ylcarbonyl)amino]ethyl}-pyrazolo[1,5-a]pyrimidine-3-carboxamide;
N-[2-(benzyloxy)ethyl]-5,7-dimethyl-6-[3-(trifluoromethoxy)benzyl]pyrazolo-[1,5-a]pyrimidine-3-carboxamide;
N-(3-amino-3-oxopropyl)-6-(3,5-dichlorobenzyl)-5,7-dimethylpyrazolo[1,5-a]pyrimidine-3-carboxamide;
5,7-dimethyl-N-{2-[(pyridin-3-ylcarbonyl)amino]ethyl}-6-[3-(trifluoro-methoxy)benzyl]pyrazolo[1,5-a]pyrimidine-3-carboxamide;
6-[4-fluoro-3-(trifluoromethoxy)benzyl]-N-[2-(2-hydroxyethoxy)ethyl]-5,7-dimethylpyrazolo[1,5-a]pyrimidine-3-carboxamide; and
6-[4-fluoro-3-(trifluoromethyl)benzyl]-N-[2-(2-hydroxyethoxy)ethyl]-5,7-dimethylpyrazolo[1,5-a]pyrimidine-3-carboxamide.

23. A pharmaceutical formulation containing a compound according to claim 21 or 22 as active ingredient in combination with a pharmaceutically acceptable diluent or carrier.

24. A pharmaceutical formulation according to claim 23 further comprising an additional active agent.

25. A method for treatment or prevention of cardiovascular diseases, obesity, non-insulin-dependent diabetes mellitus, hypertension, neurological diseases, immune disorders, cancer, essential fatty acid deficiency, acne, psoriasis, rosacea or other skin conditions caused by lipid imbalance, or for treatment of excessive hair growth, e.g. hirsutism, which comprises administering to a mammal, including man, in need of such treatment an effective amount of a compound according to any one of claims 21 or 22.

26. A method for the modulation of stearoyl-CoA desaturase activity which comprises administering to a mammal, including man, in need of such treatment an effective amount of a compound according to any one of claims 21 or 22.

27. A method for the modulation of plasma lipid levels which comprises administering to a mammal, including man, in need of such treatment an effective amount of a compound according to any one of claims 21 or 22.
